(19) United States
(12) Patent Application Publication
(10) Pub. No.: US 2023/0043407 A1
(43) Pub. Date: Feb. 9, 2023

(54) ELECTROSTATICALLY GATED NANOFLUIDIC MEMBRANES FOR CONTROL OF MOLECULAR TRANSPORT

(71) Applicant: THE METHODIST HOSPITAL SYSTEM, Houston, TX (US)

(72) Inventors: Alessandro Grattoni, Houston, TX (US); Xuewu Liu, Sugar Land, TX (US); Mauro Ferrari, Houston, TX (US)

(73) Assignee: THE METHODIST HOSPITAL SYSTEM, Houston, TX (US)

(21) Appl. No.: 17/758,644

(22) PCT Filed: Jan. 15, 2021
(86) PCT No.: PCT/US2021/013603

§ 371 Date: Jul. 12, 2022

Related U.S. Application Data
(60) Provisional application No. 62/961,437, filed on Jan. 15, 2020.
(60) Provisional application No. 62/968,670, filed on Jan. 31, 2020.

(51) Int. Cl. B81B 1/00; B81C 1/00; A61M 5/168

(52) U.S. Cl. CPC B81B 1/004; B81C 1/00087; A61M 5/16804; B81B 2201/054; B81B 2203/0353; B81B 2203/04; B81C 2201/0132; B81C 2201/0178; B81C 2201/0176; B81C 2201/0133; A61M 2205/0233; A61M 2205/3317

(57) ABSTRACT

Devices and methods for controlling molecular transport are disclosed herein. The devices include a membrane having a plurality of nanochannels extending therethrough. The membrane has an inner electrically conductive layer and an outer dielectric layer. The outer dielectric layer creates an insulative barrier between the electrically conductive layer and the contents of the nanochannels. At least one electrical contact region is positioned on a surface of the membrane. The electrical contact region exposes the electrically conductive layer of the membrane for electrical coupling to external electronics. When the membrane is at a first voltage, molecules flow through the nanochannels at a first release rate. When the membrane is at a second voltage, charge accumulation within the nanochannels modulates the flow of molecules through the nanochannels to a second release rate that is different than the first release rate. Methods of fabricating devices for controlling molecular transport are also disclosed herein.

### RELATED APPLICATIONS

This application claims the benefit of U.S. Provisional Applications 62/961,437, filed Jan. 15, 2020, and 62/968,670, filed Jan. 31, 2020. Each of the aforementioned applications is incorporated by reference in its entirety for all purposes.

### STATEMENT REGARDING FEDERALLY SPONSORED RESEARCH OR DEVELOPMENT

This invention was made with Government Support under Grant Nos. R21GM111544 and R01GM127558, both awarded by the National Institutes of Health. The Government has certain rights in the invention.

### FIELD

This invention relates to nanotechnology and microfabrication, such as they may apply to the field of drug delivery.

### BACKGROUND

Personalized care and precision medicine are emerging as important approaches for the prevention and treatment of pathologies. Patient-focused therapeutic management can be achieved by taking into account genetics, patient-to-patient variability, and environmental conditions¹. Such approaches challenge the widespread ‘one-size-fits-all’ paradigm where treatment and prevention are designed around conventional disease archetypes. Despite the substantial resources dedicated to achieving precision medicine, personalized prevention and treatment of care remain largely unmet clinical needs.

Patient-focused therapeutic management requires advanced technologies for tailored drug administration. For example, sensors are needed for constant monitoring of intra- and inter-patient variabilities to effectively achieve an individualized approach. Further, drug delivery technologies that allow for ad hoc rapid and simple adjustment of drug doses according to need represents a desirable feature. Optimally, a drug delivery system includes integration of the following factors: 1) sensing of physical or biological signals that can trigger the release, adjustment or interruption of drug release, 2) a drug delivery actuator that can continuously modulate, activate or interrupt the drug administration, 3) a feedback loop architecture that allows for further control of drug release, 4) remote communication and control capabilities to enable clinicians to adjust drug delivery independently. In the pursuit of such technology, wearable and implantable systems have gained significant interest. This is especially evident for the management of chronic diseases such as type 1 diabetes²⁻⁴, and posterior eye conditions in ophthalmology⁵, among others, where continuous monitoring and adjustment of drug doses are imperative. Along with offering long-term controlled drug delivery, implants can eliminate the widespread issue of non-compliance to treatment⁶, and pill- and treatment-fatigue. Non-adherence to chronic medications is reported at a staggering ˜50%⁷, accounting for up to 50% of treatment failures, 125,000 deaths, and approximately 25% of hospitalizations in the United States, yearly⁸. Another important consideration is that implantable systems can offer enhanced bioavailability of drugs, afford lower drug doses and hence reduce adverse effects, as well as avoid the onset of drug resistance⁹.

Considering the vast opportunities offered by autonomous “smart” delivery, numerous sensing technologies have been developed^{10,11}. Notable examples are glucose monitoring devices¹², implantable sensors for heart failure¹³, and epidermal wearable systems¹⁴, among others. Despite significant developments in sensing technologies, there is a lack of implantable drug delivery actuators that could be interfaced with sensors, for a technological platform capable of personalized patient care.

Current approaches to tunable drug delivery systems are comprised of stimuli responsive devices. These devices rely on membranes that can change the permeability of the drug upon external excitation for controlled drug release. Particle embedded membranes that respond to a magnetic field¹⁵, near-infrared irradiation^{16,17}, or ultrasound¹⁸ are notable examples. The embedded particles increase the temperature locally upon external excitation, generating a conformational change in the polymeric structure and increasing the membrane permeability. Alternatively, magnetic particles, in which the position is controlled by an external oriented magnetic field, can act as valves to open or close the membrane¹⁹. These technologies are valuable strategies for controllable drug delivery. However, they require continuous external intervention to function, which challenges their use in the next generation of autonomous drug delivery systems. Technological advances are still needed to address these issues in the field of drug delivery.

### SUMMARY

Devices for controlling molecular transport are disclosed herein. The devices include a membrane having a plurality of nanochannels extending therethrough. The membrane also includes an inner electrically conductive layer and an outer dielectric layer. The outer dielectric layer creates an insulative barrier between the electrically conductive layer and the contents of the nanochannels. At least one electrical contact region is positioned on a surface of the membrane. The electrical contact region exposes the electrically conductive layer of the membrane for electrical coupling to external electronics. When the membrane is at a first voltage, molecules flow through the nanochannels at a first release rate. When the membrane is at a second voltage, charge accumulation within the nanochannels modulates the flow of molecules through the nanochannels to a second release rate that is different than the first release rate.

Some embodiments of the devices disclosed herein include a handle layer positioned beneath the membrane. The handle layer includes at least one macrochannel extending through it. The macrochannel is fluidically coupled to the plurality of nanochannels of the membrane. In some embodiments, each nanochannel comprises an outlet on an upper surface of the membrane and an inlet connected to a macrochannel. The macrochannels can be hexagonal in shape. In some embodiments, the macrochannels are arranged in a honey-comb pattern.

In some embodiments, the height of a nanochannel, defined between a nanochannel inlet and a nanochannel outlet, is from 10,000 nanometers to 15,000 nanometers. In some embodiments, the length of a nanochannel, measured along a surface of the membrane, is from 400 nanometers to 5,000 nanometers. In some embodiments, the width of a nanochannel, measured along a surface of the membrane, is from 50 nanometers to 400 nanometers.

The dielectric layer resists degradation under physiological conditions. In some embodiments, the dielectric layer comprises a metal oxide. In some embodiments, the dielectric layer comprises silicon carbide. In some embodiments, the electrode layer comprises poly-silicon. In some embodiments, the membrane layer comprises silicon and the dielectric layer comprises silicon oxide.

In some embodiments, the edges of the dielectric layer define a gap in the dielectric layer that exposes the electrically conductive layer at the electrical contact region.

The value of a voltage applied to the membrane at the electrical contact region determines the release rate. In some embodiments, when submerged in a physiological solution, the current leakage of the device is less than 300 microamps when the voltage applied to the electrical contact region is from −1V to −3V. The device generally has ultra-low power consumption.

Methods of fabricating devices for controlling molecular transport are disclosed herein. The methods include etching a plurality of nanochannels through a membrane layer, etching a plurality of macrochannels through a handle layer positioned below the membrane layer, creating fluidic couplings between the macrochannels and the nanochannels, applying a dielectric layer to the membrane layer (thereby insulating the interior walls of the nanochannels with the dielectric layer), and forming an electrical contact region that exposes an electrically conductive surface of the membrane layer.

In some embodiments of the methods, etching a plurality of nanochannels through a membrane layer can include etching through a membrane layer from an upper surface downward to a buried oxide layer that is positioned between the membrane layer and the handle layer. Etching a plurality of macrochannels through a handle layer can include etching through the handle layer from a lower surface upward to the buried oxide layer. Fluidic couplings are created between the macrochannels and the nanochannels by removing the buried oxide layer.

In some embodiments, the membrane layer comprises a silicon electrically conductive layer, and the dielectric layer comprises silicon oxide. Other embodiments of the methods include applying an electrically conductive layer to the membrane layer (including the interior walls of the nanochannels) prior to applying the dielectric layer to the membrane layer. The electrically conductive layer can include doped polysilicon. In some embodiments, the electrically conductive layer is applied using low pressure chemical vapor deposition. In some embodiments, the electrically conductive layer is applied using ALD. In some embodiments, the dielectric layer includes silicon carbide. In some embodiments, the dielectric layer is applied by plasma enhanced chemical vapor deposition.

Some embodiments of the methods include patterning a nanochannel template onto a mask layer prior to etching a plurality of nanochannels through the membrane layer. The nanochannels can be etched using deep reactive ion etching. In some embodiments, the macrochannels are etched using deep reactive ion etching. In some embodiments the macrochannels are etched using wet etching.

In some embodiments, the electrical contact region is formed by partially removing the dielectric layer, for example, by reactive ion etching. In some embodiments, the electrical contact region is formed by masking during the deposition of the dielectric layer to the membrane layer.

Methods of controlling the delivery of a therapeutic substance through a membrane are also disclosed herein. The methods include applying a voltage to a membrane that has a plurality of nanochannels extending therethrough. The membrane also has an inner electrically conductive layer and an outer dielectric layer. The dielectric layer creates an insulative barrier between the electrically conductive layer and the contents of the nanochannels. The methods of controlling the delivery of the therapeutic substance further include inducing charge accumulation within the nanochannels extending through the membrane and modulating the rate by which a therapeutic substance is released through the nanochannels. Modulating the release rate can include releasing the therapeutic substance on an automated schedule, or releasing the therapeutic substance upon receipt of user input.

In the methods of controlling the delivery of a therapeutic substance, applying a voltage to a membrane can include applying a voltage to an electrical contact region of the membrane. The release rate can be dependent upon the value of the voltage of the membrane. In some embodiments, a voltage of −1.5V results in a release rate reduction of greater than 50%. In some embodiments, a voltage of −3V results in a release rate reduction of greater than 90%

In the methods of controlling the delivery of a therapeutic substance, the therapeutic substance can be housed in at least one reservoir adjacent to the plurality of nanochannels. The application of a voltage to the membrane then results in flow of the therapeutic substance from the reservoir through the nanochannels. In some embodiments, the at least one reservoir is a macrochannel that is fluidically coupled to the nanochannels.

### DESCRIPTION OF DRAWINGS

The device is explained in even greater detail in the following drawings. The drawings are merely exemplary to illustrate the structure and certain features that may be used singularly or in combination with other features. The drawings are not necessarily drawn to scale.

FIG. 1 is a top view of an embodiment of a device for controlling molecular transport.

FIG. 2 is a figure showing an example device for controlling molecular transport at increasing levels of magnification.

FIG. 3 is a perspective cross sectional view of a nanochannel of an example device for controlling molecular transport.

FIG. 4 is a graph showing variability in release modulation observed with applied potentials to source-drain electrodes.

FIGS. 5A and 5B are graphs showing variation in release of atenolol (FIG. 5A) and perindopril (FIG. 5B) from 50 nm nanochannels via modulation of gate potential.

FIGS. 6A and 6B show (FIG. 6A) valve architecture showing the support structure, the 15 μm-thick nanochannel layer and macro-channels; (FIG. 6B) magnification of the nanochannel with gate electrodes and SiC coating.

FIG. 7 shows scanning electron microscopy images of a microfabricated prototype of the nanochannel valve.

FIGS. 8A-8H show (FIG. 8A) Silicon On Insulator (SOI) wafer with lithography mask (FIG. 8B) Deep reactive ion etching (DRIE) for nanochannel (nCH) patterning. (FIG. 8C) DRIE for macrochannel (μCH) pattern. (FIG. 8D) SiO₂ mask removal. (FIG. 8E) SiO₂ thermal oxidation growth. (FIG. 8F) Conductive poly-Si deposition. (FIG. 8G) Insulating SiC deposition. (FIG. 8H) Membrane structure.

FIGS. 9A-9D show (FIG. 9A) Picture of the nanofluidic membrane which measure 6 mm×6 mm with a total thickness of 500 μm. (FIG. 9B) SEM image of the top face of the membrane (device layer) that shows the vertically etched nanochannels arranged in circles. (FIG. 9C) SEM image of nanochannels array. (FIG. 9D) FIB-SEM image of nanochannel cross-section which shows the vertical nanochannels and highlight the layer stack on the nanochannels walls. In order from the innermost (Silicon, blue) to the outermost layer there is silicon dioxide (SiO₂, 175 nm, green), n-doped polycrystalline silicon (poly-Si, 121 nm, red) and silicon carbide (SiC, 64.1 nm, gray). The different layers in the FIB image are artificially colored to highlight the differences.

FIGS. 10A-10I show energy-dispersive X-ray spectroscopy (EDX) for membranes coated with SiO₂ versus SiC at 77° C. (FIG. 10A), 37° C. (FIG. 10B, left-hand boxes are SiO₂ and right- and boxes are SiC at each timepoint) and at 37° C. with BSA (FIG. 10C, left-hand boxes are SiO₂ and right- and boxes are SiC at each timepoint). Surface roughness calculated with atomic force microscopy (AFM) for membranes coated with SiO₂ versus SiC at 77° C. (FIG. 10D), 37° C. (FIG. 10E, left-hand boxes are SiO₂ and right- and boxes are SiC at each timepoint) and at 37° C. with BSA (FIG. 10F, left-hand boxes are SiO₂ and right- and boxes are SiC at each timepoint). Layer thicknesses fitted through ellipsometry data for membranes coated with SiO₂ versus SiC at 77° C. (FIG. 10G), 37° C. (FIG. 10H, left-hand boxes are SiO₂ and right- and boxes are SiC at each timepoint) and at 37° C. with BSA (FIG. 10I, left-hand boxes are SiO₂ and right- and boxes are SiC at each timepoint).

FIGS. 11A and 11B show gate leakage current at different solution concentrations for SiO₂ dielectric layer (FIG. 11A) and SiC dielectric layer (FIG. 11B).

FIGS. 12A-12E show electrochemical characterization (FIG. 12A) Rendering of ad-hoc device for electrochemical measurements. (FIG. 12B) Concentration driven diffusion of negatively charged molecule. (FIG. 12C) Gated diffusion of negatively charged molecule. (FIG. 12D) Measured ionic conductance of the membrane. (FIG. 12E) Current-Voltage (I-V) curves for the membrane.

FIGS. 13A and 13B show modulated release of Alexa Fluor 647 (FIG. 13A) Rendering of ad-hoc device for in-vitro release rate modulation. (FIG. 13B) In-vitro cumulative release modulation of Alexa Fluor (top). Release rate for every phase, normalized to the average of the passive phases. Blue and red line represent the average of the passive and active (−1.5 V) phases respectively.

FIG. 14 shows modulated release of Poly(sodium 4-styrenesulfonate). In-vitro cumulative release modulation of PolySS (top). Release rate for every phase, normalized to the average of the passive phases (bottom).

FIG. 15 shows in vitro cumulative release modulation of DNA (top). Release rate for every phase, normalized to the average of the passive phases. Red line represents the average of the active (−1.5 V) phases.

FIG. 16 shows statistical analysis of release modulation. Release rates grouped by typology and compared.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

Throughout this application, various publications and patent applications are referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

“Optional” or “optionally” means that the subsequently described event or circumstance may or may not occur, and that the description includes instances where said event or circumstance occurs and instances where it does not.

As used herein, the terms “height,” when used to describe a nanochannel, refers to the distance the nanochannel extends through a membrane (from an inlet of the nanochannel to an outlet of the nanochannel). “Length” and “width” of the nanochannel are measured perpendicular to “height,” and perpendicular to each other. The “length” refers to the longer distance the nanochannel travels along a surface of the membrane, whereas the “width” refers to the shorter distance the nanochannel travels along a surface of the membrane. “Upper” refers to the side of the device including the membrane. As such, the nanochannels extend from an upper surface of the membrane to a lower surface of the membrane. In some embodiments, the lower surface of the membrane is coupled to the handle layer of the device. The terms “upper” and “lower” are for reference only (for the purposes of describing the device within this text), and are not meant to limit the orientation of the device during operation and/or implantation.

As used herein, the term “nanochannel” indicates a channel that is 1000 nanometers in width or less. The nanochannels described herein are said to extend through a “valve” or a “membrane.” The terms “valve” and “membrane” are used interchangeably in this text.

As used herein, “physiological solution” refers to aqueous salt solution, which is compatible with normal tissue by virtue of being about isotonic with normal interstitial fluid and at a physiological pH.

As used herein, “therapeutic” refers to preventing, treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition or side effect.

The devices disclosed herein control molecular transport. “Molecular transport” can include the transport of small molecules, particles, analytes, proteins, nanoparticles and/or therapeutic substances. The “release rate” is the rate by which molecules, particles, analytes, proteins, nanoparticles and/or therapeutic substances flow through the nanochannels and out the outlets of the nanochannels.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed herein is a device for controlling transport of molecules. The device leverages electrostatic gating to control transport via modulation of membrane permeability. The device can be used, for example for control of drug delivery. The device allows for continuous and reproducible dose adjustment without the need for cumbersome and bulky external triggers. It is robust and functional in a wide range of physiological conditions for timeframes that can extend over months, years or possibly decades. It lacks moving components, which could be prone to failure and limit the lifespan of the system. Further, it requires minimal energy consumption for extended function, minimizing the volume of batteries and implants. Beyond advanced functionality, this is important in the context of patient acceptability of the technology. Overall, a robust, ultra-low-power actuator technology is disclosed that is capable of efficient and reproducible control of the release of molecules. It is envisioned that when integrated with the state-of-the-art sensing technologies, the device for controlling molecular transport disclosed herein could provide a valuable solution to achieve personalized treatment of patients affected by chronic diseases.

The devices described herein can be built using microfabrication processes. As used herein, the term “microfabrication” is a concept that includes fabrication on a nanometer or micrometer level, including microfabrication and nanofabrication. Reference to certain microfabrication techniques that may be applicable in the invention can be found in Introduction to Microfabrication, Second Edition (2010) by S. Franssila. ISBN 0-470-74983-0, which is incorporated herein by reference.

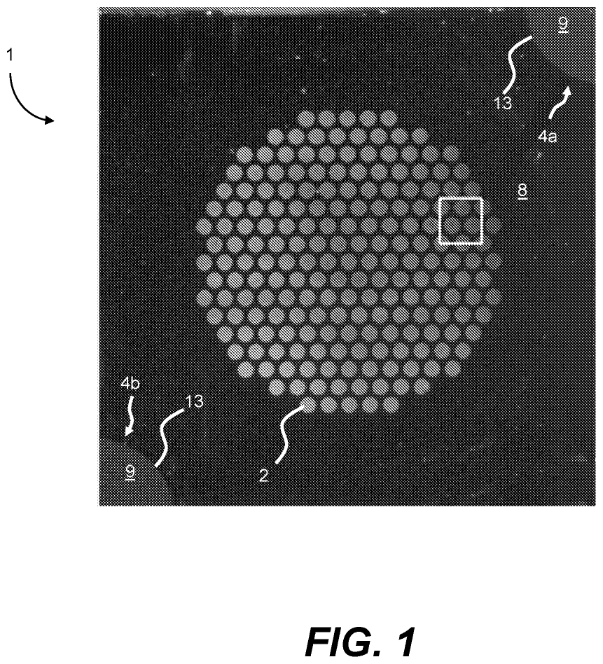

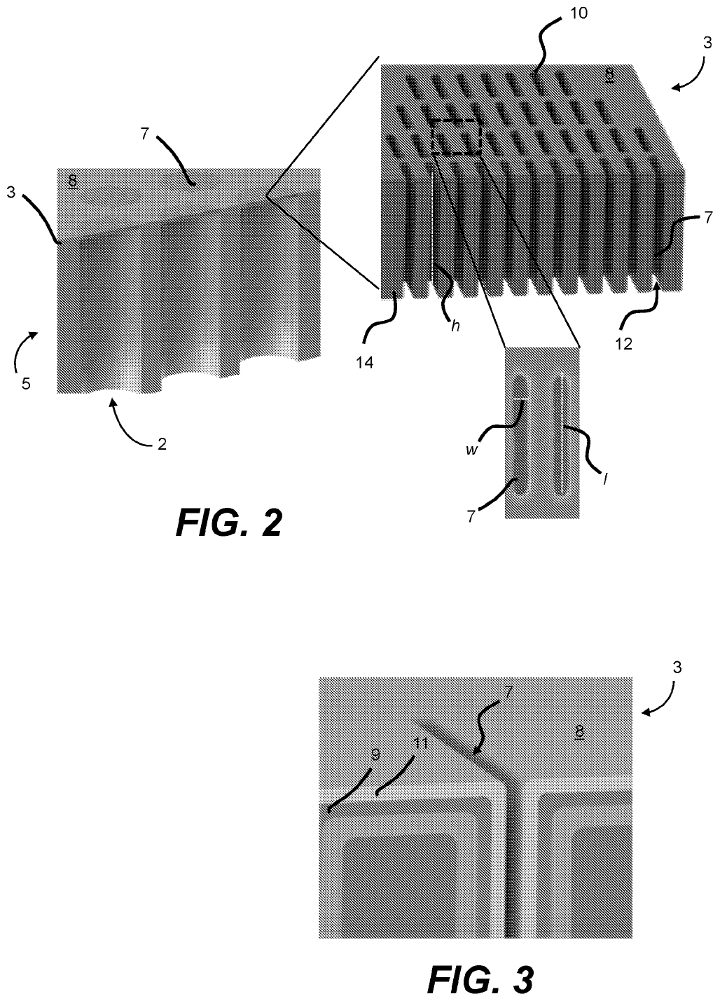

Devices for controlling molecular transport are disclosed herein. A top view of an example device 1 is shown in FIG. 1. From the top view, the macrochannels 2 are visible, as well as electrical contact regions 4a, 4b. The box drawn over a few of the macrochannels of FIG. 1 shows a region that is magnified and shown in perspective cross section in FIG. 2. The device 1 includes a membrane 3 extending along its upper surface. FIG. 2 shows aspects of the membrane 3 of device 1 at increasing levels of magnification. In some embodiments, the device includes a handle layer 5 positioned beneath membrane 3. Macrochannels 2 extend through handle layer 5 to fluidically couple with the nanochannels 7 of the membrane 3. Particularly, each nanochannel 7 comprises an outlet 10 on the upper surface 8 of the membrane 3 and an inlet 12 on a lower surface 14 of the membrane 3. The inlet 12 of each nanochannel 7 is connected to a macrochannel 2.

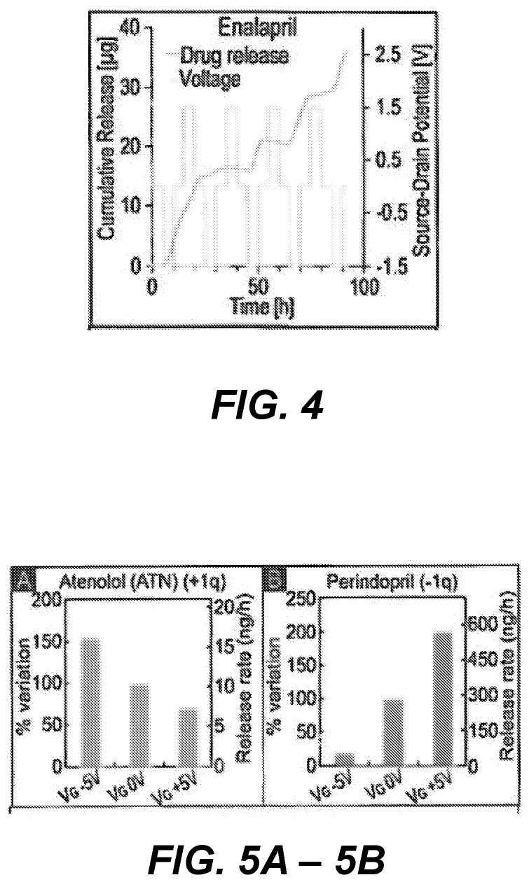

FIG. 3 shows a cross section of the membrane layer of the device 1 at the upper membrane surface 8. The membrane includes an inner electrically conductive layer 9 and an outer dielectric layer 11. The dielectric layer 11 creates an insulative barrier between the electrically conductive layer 9 and the contents of the nanochannels 7.

Referring back to FIG. 1, on the upper surface 8 of the membrane, edges 13 of the dielectric layer define a gap that exposes the electrically conductive layer 9 at electrical contact regions 4a, 4b. The electrical contact regions 4a, 4b allow the membrane 3 to be electrically coupled to external electronics. Modulation of the membrane voltage causes charges to accumulate within the nanochannels which results in changes in the flow rate of molecules through the nanochannels. In this phenomenon, charged particles electrostatically interact with the charged surfaces of the nanochannels walls creating an ionic distribution known as electric double layer (EDL). In the EDL region, depending on the sign of the surface charge, the concentration of charged molecules can either increase of decrease with respect to the bulk, to balance the surface charge and bring the nanochannel to electrostatic equilibrium. Every charged molecule that electrostatically interacts with the surface charge will experience an increase or decrease in concentration dictated by the Poisson-Boltzmann distribution of potential at the interface³⁹. For example, negative molecules will be repelled by the negative surface charge, therefore their overall concentration in the nanochannel will also be reduced. Thus, resulting in a net reduced diffusive flow with respect to a completely neutral channel. For this reason, the control over the surface charge allows the indirect control of the apparent diffusivity of charged molecules in nanoconfined space. The surface charge could in theory be increased to completely prevent a co-ion from entering the channel, creating a gate. As such, when the membrane is at a first voltage, molecules flow through the nanochannels at a first release rate, and when the membrane is at a second voltage, charge accumulation within the nanochannels modulates the flow to a second release rate that is different than the first release rate. The value of the voltage applied to the membrane determines the release rate or the rate of molecular transport through the nanochannels of the device.

In some embodiments, the first electrical contact region 4a acts as a source electrode and a second electrical contact region 4b acts as a drain electrode.

In some embodiments, the height, h, of a nanochannel, defined between a nanochannel inlet 12 and a nanochannel outlet 10 (through thickness of membrane 3), can be from about 10,000 nanometers to about 15,000 nanometers. In some embodiments, the length, l, of a nanochannel, measured along a surface 8, 14 of the membrane, can be from about 400 nanometers to about 5,000 nanometers. In some embodiments, the width, w, of a nanochannel, measured along a surface of the membrane, is from about 50 nanometers to about 400 nanometers. Length and width are defined for nanochannels that are rectangular in cross section. However, in some embodiments, nanochannels may be circular in cross section, ellipsoidal in cross section, triangular, square, pentagonal, hexagonal, or generally polygonal in cross section. In the images shown, the cross-sectional dimensions of the nanochannel are generally constant through the height/thickness of membrane 3. However, this need not be the case. In other embodiments, the cross-sectional dimensions (length, width, diameter, etc.) of the nanochannels may widen or narrow along the height, or may otherwise vary along the height due to fabrication and/or processing artifacts.

In some embodiments, each macrochannel 2 is fluidically coupled to anywhere from 1000 to 1800 nanochannels 7. In some embodiments, the macrochannels 2 are from about 300 micrometers to about 700 micrometers in height. They may or may not extend the full thickness of the handle layer 5. Macrochannels 2 can, in some embodiments, be hexagonal in a cross section taken perpendicular to the height. Hexagonal macrochannels lend strength to handle layer 5, especially when positioned in a honeycomb pattern with respect to each other. However, similar to the nanochannels, the disclosure is not meant to limit the macrochannels to any particular cross-sectional shape. Circular macrochannels 2 can be easy to fabricate. Ellipsoidal, triangular, square, pentagonal, hexagonal, or generally polygonal cross-sectional macrochannel shapes are possible and within the scope of this disclosure. In the images shown, the cross-sectional dimensions of the macrochannels are generally constant through the height/thickness of handle layer 5. However, this need not be the case. In other embodiments, the cross-sectional dimensions (length, width, diameter, etc.) of the macrochannels may widen or narrow along the height, or may otherwise vary along the height due to fabrication and/or processing artifacts. For example, macrochannels formed by deep reactive ion etching can have relatively constant cross sectional dimensions, while macrochannels formed by KOH wet etching can be the shape of a truncated pyramid.

In some embodiments, the dielectric layer 11 includes or is formed completely of a material that resists degradation under physiological conditions. Advantageously, the dielectric layer 11 can be bioinert, resisting protein adsorption and facilitating acceptance by a subject's immune system during use as an implantable device. In some embodiments, the dielectric layer 11 includes or is formed completely of a metal oxide. In some embodiments, the dielectric layer includes or is formed completely of silicon carbide. In some embodiments, the dielectric layer includes or is formed completely of, for example, silicon dioxide, titanium nitride, or conductive ultra-nanocrystalline diamond (UNCD).

The electrically conductive layer 9 facilitates the flow of charge through the membrane 3. It can be formed of any material known in microfabrication techniques to facilitate the flow of charge. In some embodiments, the electrode layer 9 comprises poly-silicon, or doped polysilicon. In some embodiments, the electrically conductive layer 9 can be formed of silicon (for example, the silicon original device layer can be coated with a dielectric layer of silicon dioxide to form a 2 layer membrane). In some embodiments, the electrically conductive layer 9 includes or is formed of an ALD-deposited conductive film such as, but not limited to, titanium or palladium.

In some embodiments, if the device is submerged in a physiological solution and a voltage is applied to the electrical contact region between −1V to −3V, the current leakage of the device is less than 300 microamps. Likewise, when submerged in a physiological solution, the device advantageously has ultra-low power consumption. Ultra-low power consumption can be, for example, less than 10 milliWatts (including less than 10 milliWatts, less than 5 milliWatts, less than 1 milliWatt, less than 750 microWatts, less than 500 microWatts, less than 250 microWatts, less 100 microWatts, less than 50 microWatts, less than 25 microWatts, and less than 5 microWatts).

Methods of fabrication are also disclosed herein. The methods include etching a plurality of nanochannels through a membrane layer and etching a plurality of macrochannels through a handle layer positioned below the membrane layer. In some embodiments, a nanochannel template is patterned onto a mask layer prior to etching a plurality of nanochannels through the membrane layer. Nanochannels can be etched, for example, using deep reactive ion etching. Macrochannels can be etched, for example, using deep reactive ion etching or wet etching.

During fabrication, fluidic couplings are created between the macrochannels and the nanochannels. In some embodiments, the device has a buried oxide layer positioned between the membrane layer and the handle layer. The nanochannels are etched through a membrane layer from the upper surface down until they reach the buried oxide layer, and the macrochannels are etched upward from a lower surface of the handle layer until they reach the buried oxide layer. The fluidic coupling of the macrochannels and the nanochannels is performed by removing the buried oxide layer.

In some embodiments, the membrane layer includes silicon (such as when the processing wafer is formed of silicon), and the silicon itself acts as the electrically conductive layer. Silicon oxide is formed on the surface of the silicon layer to form the dielectric layer. In other embodiments, an electrically conductive layer is applied to the membrane layer by a separate processing step. The electrically conductive layer is applied to surfaces of the membrane layer, including the interior walls of the nanochannels, prior to applying the dielectric layer to the membrane layer. In some embodiments, the electrically conductive layer is applied using low pressure chemical vapor deposition, or ALD. A dielectric layer is applied to the membrane layer to insulate the interior walls of the nanochannels. The dielectric layer can, in some embodiments, be applied by plasma enhanced chemical vapor deposition.

During fabrication, at least one electrical contact region is formed. This can be performed by partially removing the dielectric layer to expose an electrically conductive surface of the membrane layer, or alternatively by masking the electrically conductive layer during dielectric layer deposition, such that the dielectric layer is never applied at the electrical contact region.

Methods of controlling the delivery of a therapeutic substance through a membrane are disclosed herein, resulting in modulation of the release rate by which a therapeutic substance flows through the nanochannels. A voltage is applied to the membrane (for example, at the electrical contact regions). This results in charge accumulation within the nanochannels extending through the membrane as described above.

The release rate of the therapeutic substance can be modulated according to an automated schedule, or, in some embodiments, on demand with user input. The release rate is dependent upon the voltage of the membrane. In some embodiments, a membrane voltage of −1.5V results in a release rate reduction of greater than 50%. In some embodiments, a membrane voltage of −3V results in a release rate reduction of greater than 90%.

In some embodiments, the therapeutic substance is housed in at least one reservoir adjacent to the plurality of nanochannels. The reservoir can be, for example, the macrochannel that is fluidically coupled to the nanochannels. Application of a membrane voltage can result in flow of a therapeutic substance from the reservoir through the nanochannels.

### EXAMPLES

Patient-centered therapeutic management for chronic medical conditions is a desired but unmet need, largely attributable to the lack of adequate technologies for tailored drug administration. While triggered devices that control the delivery of therapeutics exist, they often rely on impractical continuous external activation. As such, next generation continuously tunable drug delivery systems independent of sustained external activation remain an elusive goal.

Microfabricated devices containing gated nanochannels are disclosed herein, and can be used to electrostatically control the transport of molecules in a fluid environment. Gate electrodes, buried underneath the sidewalls of nanochannels, allow for the electrostatic tuning of the nanochannel surface charge to alter the distribution of ions and other species within the fluid contained in the nanochannels. The electrostatic modulation of charge distribution can be adopted to modify the rate of diffusive, convective, or electrokinetics transport of charged molecules and particles across the valve and obtain an increase, decrease, interruption, or reactivation of the rate of molecular transport. Applications range for fluid filtration, lab on a chip diagnostic systems, energy generation, drug delivery, and particle separation.

### Example 1: Gated Nanofluidic Valve for Active and Passive Electrosteric Control of Molecular Transport

Background and the Choice of Gate-Electrodes:

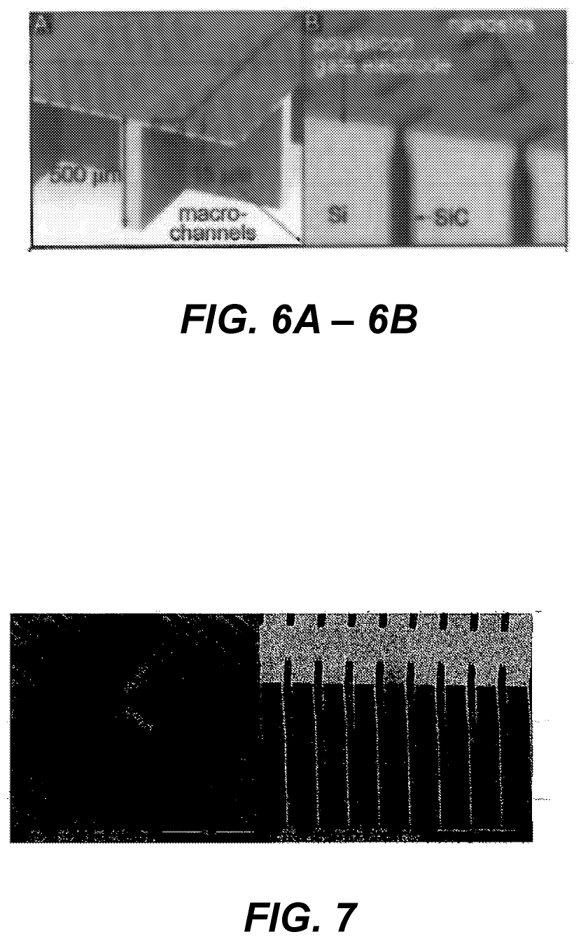

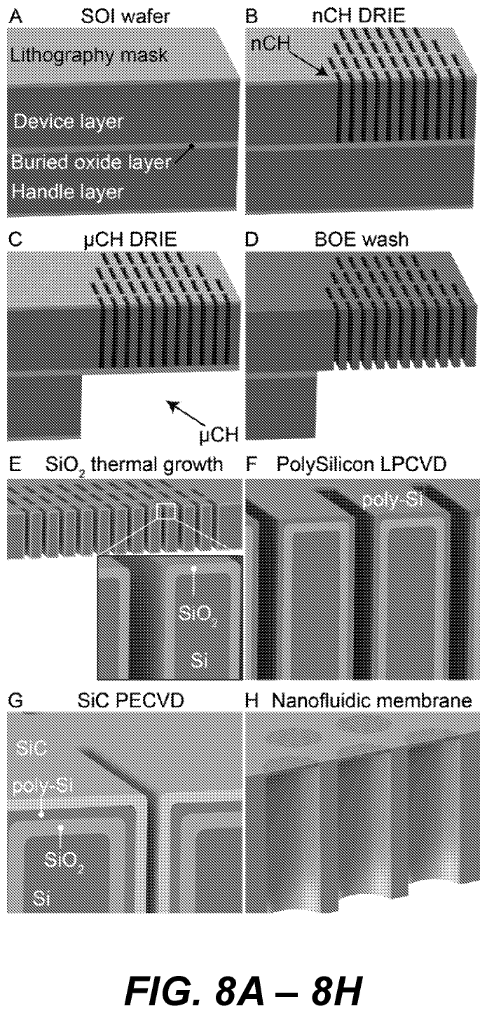

In this work the use of an applied source-drain electrical field across nanochannel membranes to modulate drug delivery from a reservoir was explored. Existing nanochannel membranes have been modified by adding source and drain electrodes on their surface, created custom testing apparatuses to test controlled release of drugs in vitro, and developed various prototypes of implant reservoirs. In vitro drug release studies showed that an applied source drain potential was able to modify drug release from a reservoir. However, such control was not always reproducible and results were subject to variability (FIG. 4). This was likely due to the need for uninsulated electrodes exposed to the accumulation of ionic species and thus subject to progressive charge screening. Further, substantial power consumption was measured, limiting the system's autonomy, or requiring the use of larger batteries. As these limitations were realized, it was discovered that electrostatic gating in nanochannels would better offer an ideal control strategy. As a proof of concept, nanochannel constructs with gate electrodes were developed and the modulation of drug release via applied gate potentials was tested. Electrostatic gating showed to be reproducible and effective in fine tuning drug release, enabling both an increase and decrease in drug delivery (FIGS. 5A-5B). Modulation was associated with ultra-low power consumption, ideal for implantable applications. The high energy efficiency and reliability of electrostatic gating can be ascribed to the use of fully insulated gate electrodes, which “gate” drug delivery by simply modulating the charge of the nanochannel and the electrical double layer developed within.

### Nanofluidic Valve Design Example 1

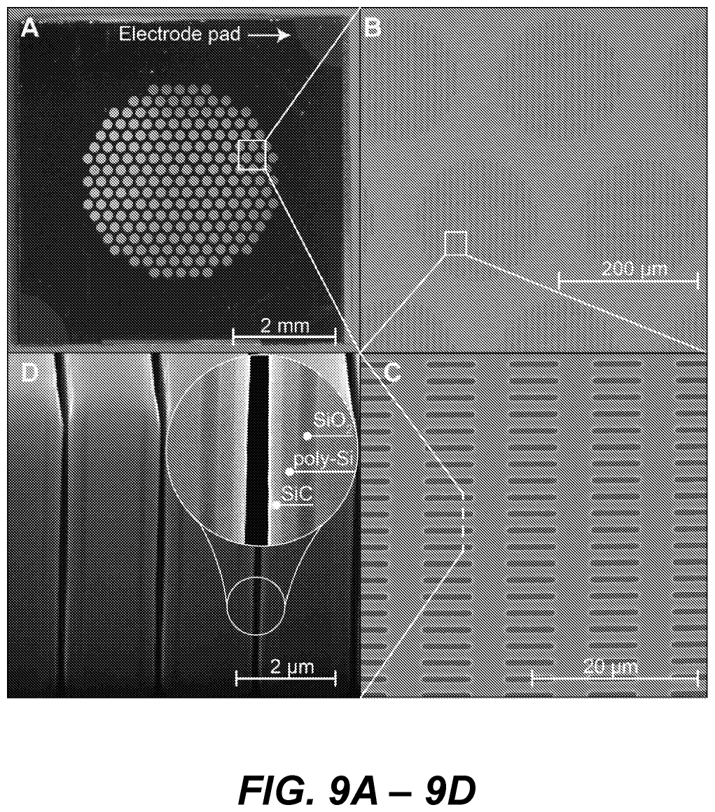

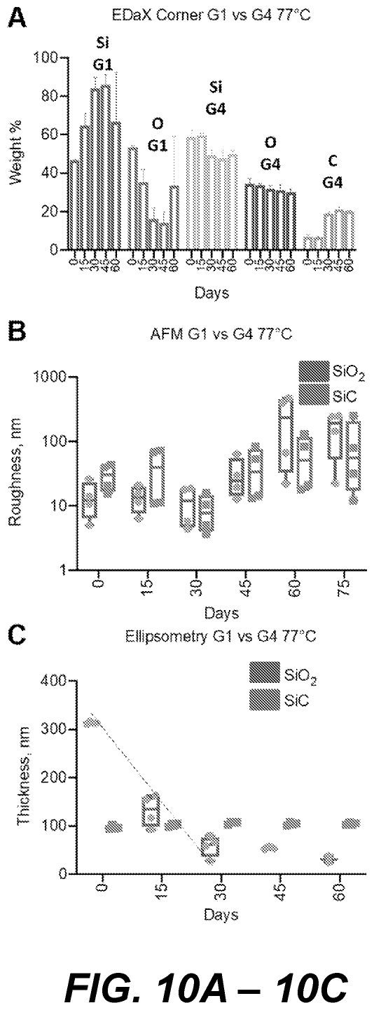

FIGS. 6A-6B show an example of a 500 μm-thick valve architecture presenting a support structure housing mesh of square macro-channels (500 μm length) and a 10 μm-thick nanochannel layer. These dimensions are presented as an example only; other sizes are possible. This structure provides a nanochannel valve with significant mechanical robustness. 50 nm wide nanochannels (3 μm length, 10 μm height) are fabricated in dense arrays (FIGS. 6A-6B). The nanochannel size (50 nm) was selected based on proof of concept studies with gate electrodes as a workable channel size for the modulation of drug release through an applied gate potential. However, nanochannel size can be easily modified during the microfabrication process in the range from 10-1,000 nm the. Each membrane contains a precise number of nanochannels (exactly 687,280 nanochannels in the example described here) incorporating gate electrodes. However, the number of nanochannels can also be varied. FIG. 7 shows scanning electron microscopy images of a microfabricated prototype of the nanochannel valve.

Potential Design Parameters:

The dimensions of a nanochannel valve have profound effects on its function. The width and ratio of width to height of the nanochannels contribute to the drug release profile and power-off release rate. The width of the nanochannels also affects the voltage needed for gated control. The dimension of template nanochannels can be a wide range, from 10s nm to micron depending on the limit of lithography tools. The aspect ratio of deep silicon etch tools is also a parameter to consider. 400 nm is the limit of an contact aligner. Using advanced photolithography tools, as little as 10 s nm features can be patterned. However, the aspect ratio of deep silicon etching limits the possible height of nanochannel valve. For example, nanochannels with a width of 400 nm and a height of 15 μm have also been fabricated.

The layout of supporting mesh (handle layer) contributes to the mechanical strength of the valve, Square mesh is a typical supporting structure, and can be fabricated by using ICP deep silicon etching. Truncated pyramid shape macrochannels with sloped sidewalls can be readily fabricated through KOH based wet etching. Hexagonal macrochannels, arranged in a honeycomb pattern, provide good mechanical stability. However, circular macrochannels may be easier to fabricate from a production standpoint.

Potential Fabrication Method 1:

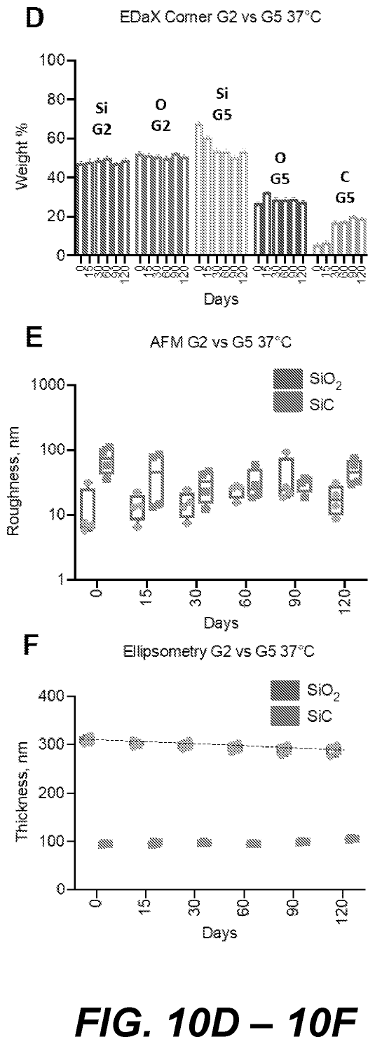

To fabricate the structure mentioned above, 4 inch SOI wafers are used (15 μm device layer with 500 μm thick handle wafer, in this example). FIGS. 8A-8H provide an exemplary schematic of an example fabrication process (with FIGS. 8A-8G showing steps of an exemplary process, and FIG. 8H showing a zoomed-out view of the final or near-final product). FIGS. 8A-8H will be described in more detail in Example 2, below. In this example, template nanochannels (400 nm width, 5 μm length) are patterned on the device layer using standard photolithography on a contact aligner (SUSS MAG), and nanochannel patterns are etched through the 15 μm device layer via ORIE on ICP etcher (Plasma Therm Versaline), and stopped at the oxide layer of SOI (later removed by HF). The macrochannel mesh is then patterned on the backside of SOI using backside alignment on aligner (SUSS MAG), ICP deep silicon etching is carried out to etch through the 500 μm handle wafers, and stopped at the oxide layer of SOI. After cleaning the polymer built up on the sidewalls, oxide layer of SOI is removed in HF to connect nanochannels and macrochannels. Then a 50 nm oxide layer is grown all over the surface as insulating layer. To fabricate the gate electrodes, doped polysilicon is deposited (75 nm thickness) via LPCVD, creating a reduction of the cross section at the nanochannel inlets. As polysilicon deposition is slow, this reduction is tightly controlled and allows us to generate channels with uniform dimensions. The gate electrode and whole structure are coated via CVD by a 50 nm silicon carbide (SiC) dielectric layer, which provides excellent coating uniformity and bioinertness. Other coatings (e.g. TaN or conductive Ultra-Nanocrystalline Diamond (UNCD), for example) and strategies to generate the gate electrodes are available. Highly doped silico wafers can be used as gate electrode in conjunction with an isolation layer. The valve will present an electric contact pad in a corner of the front side surface for connecting to control electronics. Finally, wafers are diced into individual valves.

Potential Fabrication Method 2:

Alternatively, mechanical supporting macrochannels can be truncated pyramid shaped holes for a smaller area valve, and achieved by KOH wet etching. To fabricate this structure, 4 inch SOI wafers are used (15 μm device layer with 500 μm thick handle wafer). Template nanochannels (400 nm width, 5 μm length) are patterned on the device layer using standard photolithography on a contact aligner (SUSS MA6), and nanochannel patterns are etched through the 15 μm device layer via DRIE on ICP etcher (PlasmaTherm Versaline), and stopped at the oxide layer of SOI (later removed by HF). Then the macrochannels are patterned on the backside of SOI using backside alignment on aligner (SUSS MA6). KOH wet etch (40% KOH, 80° C.) is carried out to etch through the 500 μm handle wafers, and stopped at the oxide layer of SOI. Sloped walls (54.7 degree) are typical feature of KOH etching. Oxide layer of SOI is removed in HF to connect nanochannels and macrochannels. Then a gate electrode is fabricated as discussed in potential fabrication method 1.

Potential Fabrication Method 3:

Alternatively, instead of deposited polysilicon, the highly doped silicon device layer can be used as gate electrode, and thermal oxide grown with accurate thickness can be used to define the nanochannel width and also as insulation. To fabricate the structure, 4 inch SOI wafers are used (15 μm doped silicon device layer). Template nanochannels (400 nm width, 5 μm length) are patterned on the device layer using standard photolithography on a contact aligner (SUSS MA6), and nanochannel patterns are etched through the 15 μm device layer via DRIE on ICP etcher (PlasmaTherm Versaline), and stopped at the oxide layer of SOI (later removed by HF). Then the macrochannels are opened. After removing oxide of SOI, thermal oxide is deposited to conformal insulation layer and shrink the nanochannels to desired dimension.

Potential Fabrication Method 4:

Alternatively, atomic layer deposited (ALD) conductive film can be applied as gate electrode, such as ALD titanium, or palladium. ALD Titanium nitride can also be followed to coat the metal film as insulator.

Results:

In pilot studies, gated valves were fabricated presenting 50, 100 and 150 nm wide channels. The application of the gate potential was most effective in 50 nm channels and generated a significant and reproducible change in the rate of drug release from the nanochannels via an applied ±5 VDC gate potential (FIGS. 5A, 5B).

Problem Addressed and Advantages:

This resolves the problem of the low power control of molecules and particle transport. Current approaches using electrokinetics systems or electromechanical devices are complex and require substantial amounts of energy. The energy consumption of this valve system is virtually negligible, which will be ideal for numerous applications including energy production, large scale water filtration, and implantable drug delivery systems. The nanochannel valve presents at least three advantages over existing technology: 1) Electrostatic gating of nanochannels: the gate electrode nanochannels control the transport of charged molecules and particles through electrosteric modulation (nanoconfinement and electrostatic gating). In the case of passive, concentration-driven diffusion (no applied gate potential), the valve achieves a controlled constant transport rate. In the case of an applied electrical potential at a gate electrode, an ionic redistribution occurs within the nanochannels effectively enhancing or gating the transport of molecules, effectively and reproducibly modulating or interrupting their release. 2) Ultra-low power consumption: by adopting isolated gate electrodes the transport modulation occurs with negligible electrical current (leakage currents). Power consumption is minute (nA). 3) Versatility: The valve is versatile as it allows for the control of transport of a wide spectrum of molecules including small molecules, proteins and nanoparticles. Importantly, the nanochannel valve will be affordable. The microfabrication allows for parallel manufacturing and is inexpensive. Therefore, this device offers widespread utilization and has broad applicability.

### Example 2: Electrostatically Gated Nanofluidic Membrane for Ultra-Low Power Controlled Drug Delivery

Introduction: A silicon carbide (SiC)-coated membrane featuring a buried doped polysilicon electrode was developed using fabrication techniques derived from the semiconductor industry.⁴⁵ The electrode extends under the whole surface of densely packed nanochannels. The SiC dielectric layer acts as an electrode insulator providing low leakage currents, thus reducing energy loss. Further, it provides biocompatibility and chemical inertness for extended use as implantable system. In this example, membrane bioinertness is characterized in simulated in vivo conditions at 37° C. and under accelerated testing at 77° C. To evaluate energy consumption, the SiC insulation is characterized in comparison to commonly used SiO₂. Finally, in vitro release rate modulation is demonstrated for two charged model molecules: Alexa Fluor 647 and poly(sodium 4-styrenesulfonate). Overall, this example provides the proof-of-concept of a robust, ultra-low-power actuator technology capable of efficient and reproducible control of the release of molecules. It is envisioned that when integrated with the state-of-the-art sensing technologies, the gated membrane could provide a valuable solution to achieve personalized treatment of patients affected by chronic diseases.

Materials and Methods

Nanofluidic Membrane Fabrication:

The membranes employed in this study were fabricated starting from a 4-inch p-doped silicon-on-insulator (SOI) substrate with a device layer (10 μm), a buried oxide layer (1 μm) and a handle wafer (400 μm; Ultrasil Corporation, Hayward, Calif.). Exemplary fabrication steps are illustrated in FIGS. 8A-8H. First, a 600 nm thermal oxide was deposited on the surface of the SOI wafer to act as mask layer for photolithography (FIG. 8A). Arrays of template nanochannels (500 nm width by 6 μm length) were patterned on the device layer by using standard photolithography on a contact aligner (SUSS MA6). After transferring the pattern into the oxide mask layer by reactive ion etching (RIE), nanochannel patterns were etched through the 10 μm device layer via deep RIE (DRIE) on an ICP deep silicon etcher (PlasmaTherm, Versalline), and stopped at the middle oxide of the SOI (FIG. 8B). On the other side of the SOI, the handle wafer was patterned using double side alignment on the aligner (SUSS MA6). The layout of the handle wafer was designed with a high density of hexagonally arranged circular macrochannels to provide mechanical stability. ICP deep silicon etching was used to etch through the 400 μm handle wafer, stopping at the buried oxide layer (FIG. 8C). After cleaning the polymer build up on the sidewalls of nanochannels and macrochannels, the buried oxide layer of the SOI was removed in a buffered oxide etchant (BOE) solution to connect the nanochannels and macrochannel mesh (FIG. 8D). The resulting nanochannels have an average height of 770 nm. Following that, a wet thermal oxidation was performed at 1055° C. in ultra-high-purity (UHP) water vapor for 11 min, resulting in a high temperature oxide (HTO) SiO₂ formation that shrinks the nanochannel height to 580 nm (FIG. 8E). As the thermal oxidation is a slow process, the nanochannel size reduction can be tightly controlled, allowing the generation of channels with defined dimensions. To form the gate electrodes, phosphorus doped polysilicon (poly-Si) was deposited (120 nm thickness) via low-pressure chemical vapor deposition (LPCVD; FIG. 8F). The whole wafer structure was coated with a 64 nm SiC dielectric layer via plasma-enhanced chemical vapor deposition PECVD (FIG. 8G). SiC forms an excellent bio-inert coating, while serving as an insulating layer for the gate electrodes. To expose the highly doped poly-Si, two contacts pads (˜1 mm²) were created at the edge of the membranes by selective removal of SiC by fluorine-based RIE.

Each wafer features 120 membrane chips, which were diced into individual membranes (6×6 mm) via a dicing Saw (ADT 7100 Dicing Saw). Each 6 mm by 6 mm chip presents 199 round macrochannels organized in a hexagonal spatial configuration (FIG. 8H). Every macrochannel is connected to 1400 identical slit nanochannels organized in 19 rows and 96 columns. Each membrane chip features a total of 278,600 nanochannels.

Membrane Degradation:

To test the durability of the membrane, an in vitro study was performed in simulated physiological conditions at 37° C. as well as in accelerated conditions at 77° C. Two sets of membranes were employed: 1) in the first set, the fabrication procedure was stopped at the thermal oxidation (Set A), thereby resulting in the outmost layer of SiO₂ (˜300 nm), 2) the second set of membranes (Set B) featured an outmost layer of SiC (˜70 nm), which was deposited as previously described right after SiO₂ (˜270 nm) thermal growth. Each set of membranes was divided into 3 groups: the first group was soaked in 4 mL of 2 μM sodium fluoride (NaF) in PBS at 77° C., the second group was soaked in the same solution at 37° C. and the third group in 2 μM NaF in PBS with 16 mg/mL BSA at 37° C. This resulted in a total of 6 groups with N=4 for each. To prevent exposure of SiO₂ from the side in the Set B, the sides of each membrane were covered with thermal epoxy (354-T Epoxy Technologies, Inc.) and cured at 150° C. for 30 minutes.

The degradation study was run for a total of 120 days with timepoints every 15 to 30 days depending on the group. At each timepoint, the membranes were removed from the solution and triple rinsed in deionized water (DI H₂O) followed by isopropyl alcohol (IPA) before being dried. Surface roughness (AFM Catalyst), surface composition (EDAX, Nova NanoSEM 230) and thickness of the different layers (J. A. Woollam M2000U ellipsometer) were measured to assess degradation.

Focused Ion Beam (FIB), Scanning Electron Microscope (SEM) Imaging:

The structure and fabrication repeatability of the nanofluidic membrane was assessed by imaging with a dual-ion beam (FIB) system FEI 235 at the nanofabrication Facility of the University of Houston, Tex. Nanochannel cross sections were obtained using gallium ion milling. The resulting structures where imaged at a 52° angle using scanning electron microscopy (SEM).

Ellipsometry Measurements:

The thickness of the different layers composing the membrane was measured with a multiangle spectroscopic ellipsometer (J. A. Woollam M2000U).

Electrode Connection:

Insulated high-temperature 36 AWG wires (9510T1, McMaster Carr, Douglasville, Ga.) were connected to the exposed contact using conductive silver epoxy (H20E, Epoxy Technology, MA) and cured at 150° C. for 1 hour. The conductive contact was then isolated with UV epoxy (OG116, Epoxy Technologies, Inc.) and cured with a UV lamp (UVL-18, UVL) for 2 hours.

Dielectric Leakage Current:

Gate leakage studies were performed in a custom made two reservoir fixture made of transparent Poly(methyl methacrylate) (PMMA) (McMaster Carr, Douglasville, Ga.). Each reservoir contains 2 mL of solution. The membrane under testing was sandwiched between the two reservoirs by means of two silicon rubber O-rings (Apple Rubber, Lancaster, N.Y.). The entire assembly was secured together by 4 SS316L M3 screws. Each reservoir contained two Ag/AgCl electrodes. Both reservoirs were filled with either 1×PBS, 0.1×PBS or 0.01×PBS solution. The voltage was applied between the gate electrode (Working Electrode) and the two Ag/AgCl electrodes (Counter and Reference Electrodes) in the reservoir facing the nanochannels using an electrochemical workstation (CH Instruments, Inc. 660E). A staircase of 250 mV steps was applied from −3 V and +3 V. Each step was hold for 30 s to overcome transient phenomena.

Conductance and I-V Curves:

Conductance and I-V curves were performed in the same two reservoir fixtures previously described for the leakage current. Conductance measurements were performed with a 4-electrode configuration, two for each side of the membrane. KCl solution was employed with concentrations ranging from 0.1 μM to 100 mM. The solution in both reservoirs was changed after each measurement. The voltages were applied using an electrochemical workstation (CH Instruments, Inc. 660E). A staircase of 250 mV steps was applied from −1.5 V and +1.5 V. Each step was hold for 30 s to overcome transient phenomena. The conductance (measured as current measured divided by voltage applied) was calculated for each applied voltage and averaged. The same membrane was tested 3 times. Three different membranes were tested using the same procedure. No gate voltage was applied during conductance measurements.

Current-voltage (I-V) curves were performed with the same two reservoir fixture previously described. In this case though, one reservoir featured 3 Ag/AgCl electrodes. A 10 μM KCl solution was employed in both reservoir that was refreshed after every measurement. Voltages across the membrane (V_{DS}) were applied using an electrochemical workstation (CH Instruments, Inc. 660E) in 4-electrode configuration. A staircase of 250 mV steps was applied from −1 V and +1 V. Each step was hold for 30 s to overcome transient phenomena. The gate voltage was applied between the gate electrode and the Ag/AgCl electrode in solution using an electrochemical analyzer (CH Instruments, Inc. 621D). A constant voltage of either −1.5 V, 0 V or 1.5 V was applied and the current monitored.

Both measurements were performed on membranes with a poly-Si buried electrode and a SiC insulating layer with a nanochannels size of ˜300 nm.

In Vitro Release Fixture:

Release modulation experiments were performed with a custom made, two reservoirs fixture comprising of a macro cuvette (sink reservoir) and a drug reservoir. The cuvette was glued with UV epoxy (OG116, Epoxy Technologies, Inc.) to a PMMA (McMaster Carr, Douglasville, Ga.) membrane holder. The drug reservoir (500 μL capacity), made of PMMA, was secured to the membrane holder through 2 SS316L M3 screws. The membrane under testing was clamped between the two PMMA pieces, with 2 O-rings (2418T113, McMaster Carr, Douglasville, Ga.) to avoid solution leakage. The reservoir was capped with a silicone plugs (9277K87, McMaster Carr, Douglasville, Ga.).

In Vitro Release Modulation:

Release modulation experiments were performed using 300 nm membranes with both poly-Si and SiC deposition. After the electrode connection, membranes were immersed in isopropyl alcohol for 1 h to ensure proper channel wetting, rinsed in deionized H₂O at least three times and immersed in a sink solution of 0.01×PBS overnight. After filling the sink reservoir with 4.45 mL of 0.01×PBS solution, the membranes were assembled in the diffusion fixture. The source reservoir of the diffusion fixture was loaded with either 300 μl/mL in 0.01×PBS Alexa Fluor 647 (Thermo Fisher Scientific, Waltham, Mass.) (N=1) or 200 μg/mL in 0.01×PBS of Poly(sodium 4-styrenesulfonate) (243051-5G, Sigma Aldrich, St. Louis, Mo.) (N=4). At pH 7.4, both molecules are negatively charged, −3q (=−4.8×10⁻¹⁹ C) for Alexa Fluor and ˜−380q (=−608×10⁻¹⁹ C) for Poly(sodium 4-styrenesulfonate). A reference Ag/AgCl pellet electrode (Harvard Apparatus, Holliston, Mass.) was positioned in the source reservoir.

The assembled diffusion fixtures were then loaded in a robotic carousel²⁰, which is connected to a Cary 50 UV-vis spectrophotometer (Agilent Technologies). Absorbance measurements of the sink reservoir were automatically performed every 5 minutes. Between each measurement, the sink solution was under constant stirring to ensure sample homogeneity. Wavelengths used for detection were 647 nm for Alexa Fluor and 256 nm for Poly(sodium 4-styrenesulfonate). Electrical DC potentials were applied between the reference and the gate electrode using an arbitrary waveform generator (Keysight Technologies 33522A) in a succession of passive (0 V) and active (−1.5 V or −3 V) phases. Phase durations were 12 h and 8 h for passive and active, respectively.

Statistical Analysis:

Graphs were plotted and statistical data analyses were performed with GraphPad Prism 8 (version 8.1.1; GraphPad Software, Inc., La Jolla, Calif.). Data are represented as mean±SD. Statistical significance was determined using paired t-tests. For statistical analyses, the cumulative release of each phase was fitted with a first order polynomial using MATLAB™ polyfit function. The resulting angular coefficient represent the release rate of the considered phase.

Results

Nanofluidic Membrane:

To assess the quality of the fabrication process, all chip membranes were first visually inspected through optical microscopy. Gas test characterization was then performed on all chips to assess the nanochannel dimension uniformity across the wafer. A previously developed model was employed to predict the nanochannels dimension from the measurement of nitrogen gas flow through the membrane when a pressure difference is applied²¹. Chips at the edge of the wafer were excluded due to known fabrication challenges such as etching uniformity across large areas. As a result, wafer yield of 40% was achieved with nanochannel size that had a maximum variation from the expected value (300 nm) of 16%. A Gaussian non-linear fit (R²=0.99) of the cumulative distribution of the obtained values shows a predicted nanochannel size of 292±44 nm.

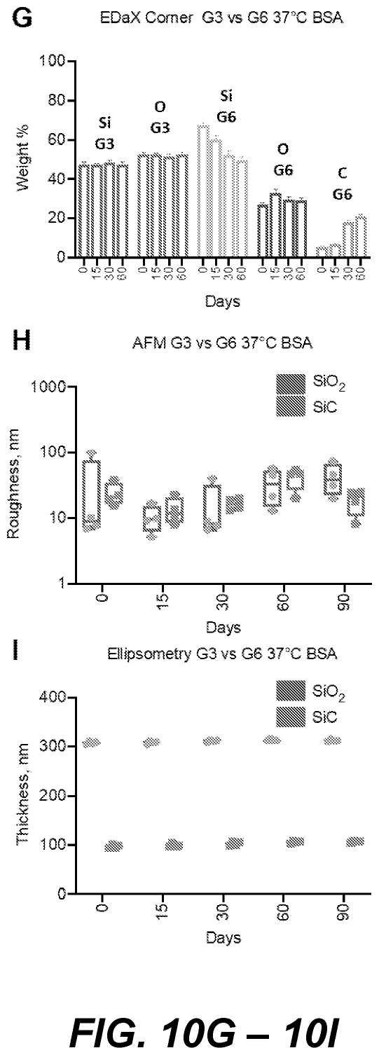

Selected chips were further analyzed using FIB-SEM imaging (FIGS. 9A-9D). FIG. 9A shows a picture of a single diced chip which has a size 6 mm×6 mm and a thickness of 400 μm. The membrane features 199 cylindrical macrochannels which measure 200 μm in diameter and 390 μm in length. The hexagonal configuration of the cylindrical macrochannel ensures high channels density and mechanical robustness for the membrane structure. Nanochannels are efficiently aligned in a circular pattern fill the macrochannel area to which they are connected (FIG. 9B, 9C).

To closely examine the obtained nanochannel dimension and the layer depositions on the channel walls, cross sections of the nanochannels were created using a gallium focused ion beam (FIB) (FIG. 9D). The slit nanochannels result in a 10 μm length and 6 μm width. Despite the high aspect ratio, it was possible to achieve nanochannels with high uniformity (FIG. 9D). The innermost SiO₂ layer created via slow thermal oxidation allows for tight control of the nanochannels dimension. The poly-Si is used as a distributed gate electrode that extends for the whole nanochannels area to offer high electrostatic gating performances. External connection to the poly-Si layer is possible through the conductive pads at the edge of the chip (FIG. 9A). The outer-most layer of SiC forms an excellent bio-inert coating, while serving as an insulating layer for the gate electrodes. As the SiC deposition is performed on both sides of the wafer, a slightly thicker layer of SiC can be noted at the entrance and exit of nanochannels due to the limited diffusivity of precursor gases in nanoconfinement during deposition. This slight non-uniformity is not expected to decrease the performance of the membrane, instead, it can potentially increase it. In fact, as the nanochannel narrows, the electrostatic effect on charged particles increases, resulting in a more pronounced gating effect.

The present membrane presents two key advantages over previous devices: i) the streamlined fluidic structure, with cylindrical microchannels directly connected to the array of through nanochannels allows for a substantially simplified fabrication process; ii) by accounting for same nanochannel size, the fluidic architecture achieves a 45% and 37% reduction in diffusive length and resistance, respectively. In other AAO-based gating systems, dispersed pores size can affect performances; by contrast, the present membrane possesses monodispersed channel dimensions. This facilitates tight control of drug delivery. Further, in contrast to most gated fluidic systems, designed for the evaluation of electrostatic phenomena, this technology achieves molecular transport rates suitable for medical applications.

Degradation Study:

In vitro degradation testing was performed to evaluate the membrane chemical robustness in view of its application for implantable drug delivery. The testing conditions in PBS at 37° C. were chosen as they represent an established model of biological fluids in subcutaneous tissues. Accelerated conditions at 77° C. allowed for the monitoring of long-term degradation within a shorter timeframe, while maintaining relevance with respect to the physiologic conditions. It is important to assess the structure integrity of the nanofluidic membrane over time because the structural integrity is related to the reliability of the gating. Phosphate buffer saline (PBS) was used to simulate the interstitial fluid at physiological conditions for all groups. Moreover, to recreate the worst possible conditions for a silicon substrate, sodium fluoride (NaF, 2 μM) was added in all groups because fluoride ions are known to be etchants of silicon dioxide. Humans are exposed to small amounts of fluoride usually through dietary intake, respiration and fluoride supplements. Additionally, because it is not homeostatically regulated, fluoride concentration in human plasma can vary widely, but rarely exceeds 0.06 ppm²² which converts to a concentration of 1.43 μM. Therefore, the inclusion of fluoride ions in the degradation studies attempts to simulate a true physiological environment. The 2 μM concentration of NaF was a conservative choice given it is greater than the high end of physiological concentration (1.43 μM).

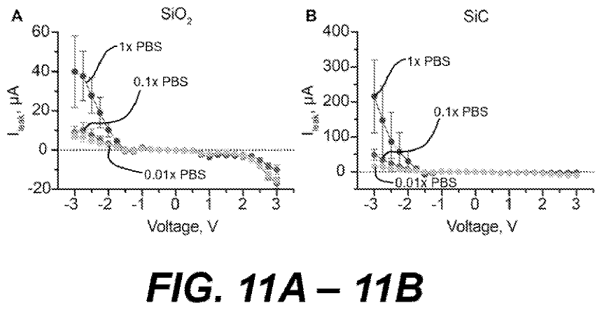

The surface composition of the chips was analyzed through energy dispersive X-ray spectroscopy (EDX). For SiO₂ chips at in accelerated conditions, the relative concentrations of silicon and oxygen significantly changed during the first 30 days, resulting in an increasing trend of silicon presence (FIG. 10A). The surface composition of the SiO₂ was not expected to change with time, but the initial thin layer (˜300 nm) of SiO₂ eroded in the solution, affecting the average volumetric composition of the surface. In fact, the EDX which usually has a depth of 1-2 μm, also includes energy from the silicon wafer underneath the thin layer, skewing the overall concentration toward silicon. The constant surface roughness (FIG. 10B) hints that the surface composition at the solid liquid interface did not change. However, ellipsometry measurements (FIG. 10C) evidently show a sustained decrease in silica thickness. Interpolation of the ellipsometry data in the first 30 days results in a calculated 8.5 nm/day dissolution of silica. Following this prediction, the 300 nm of SiO₂ has probably been completely corroded within the first 35 days. Therefore, measurements at subsequent timepoints (45, 60 and 75 days) are in fact of the underlying silicon (Si) of the device layer. This hypothesis is corroborated by the increased surface roughness observed at these timepoints. Additionally, both the ellipsometry and EDX measurement at the 45 and 60 days timepoints still show the presence of oxygen on the surface which can be explained by the formation of Si—O—Si bonds that occurs due to nucleophilic attack of oxygen from OH-terminated Si to nearby surface Si atoms with dangling bonds²³. The remaining silicon surface therefore is concurrently oxidized and hydrolyzed by the surrounding water. Due to the increased speed of hydrolysis of pure silicon with respect to SiO₂, the resulting surface roughness is increased (FIG. 10B) at these last timepoints. These phenomena help explain the presence of oxygen on the surface, even though the whole layer of SiO₂ has already been corroded.

Turning the focus to the silicon chips with SiC coating, no significant trends were seen in any of the measurements performed (FIGS. 10A, 10B, and 10C). However, there is an abrupt increase of carbon concentration of the surface composition (FIG. 10A) starting from the 30 days timepoint, which is compensated by a reduction in silicon. Being an abrupt change, rather than a continuous one, this may be related to experimental error in the fitting of the raw EDX spectrum. Nevertheless, it is remarkable how the thickness of SiC, measured with ellipsometry (FIG. 10C), appears consistent and constant over the whole duration of the experiment. This demonstrates the high inertness of SiC in electrolytic solutions, even in the presence of fluoride ions.

When examining the results at 37° C. (FIG. 10D, 10E, 10F) no significant trends were noted for the surface composition (FIG. 10D) or the surface roughness (FIG. 10E). However, a mild decreasing trend was seen in the SiO₂ thickness. The interpolation of these values results in a silica degradation rate of 0.17 nm/day. To correlate the two results (at 37° C. and in accelerate conditions at 77° C.) a temperature coefficient Q₁₀ and the Arrhenius equation t_acc=t_test Q_10\hat{ }((T−37)/10)²⁴ can be used. As a “rule of thumb”, the temperature coefficient for most corrosion reactions is about 2-2.3. However, this coefficient can both change with the specific involved materials and temperature range²⁴. In fact, with NaF in solution, it was found that between 37° C. and 77° C. the temperature coefficient Q₁₀ is 2.65, resulting in a factor of 50 between the two experimental conditions.

Additionally, the influence of protein adsorption (bovine serum albumin BSA) onto the silicon chip on the degradation was investigated (FIG. 10G, 10H, 10I). It was found that proteins in solution effectively decelerate the surface degradation. In fact, even for SiO₂, no decreasing trend in surface thickness can be appreciated from the ellipsometry data (FIG. 100. This effect may be related to the adsorption of albumin on the silica surface which effectively limits the diffusive access of water to the surface and the subsequent degradation²³.

Overall, silicon carbide (SiC) was found to exhibit high resistance to oxidation by fluoride ions. In fact, even at 77° C., SiC did not show any sign of surface dissolution in the ellipsometry data (FIG. 10C). On the other hand, SiO₂ showed an increased degradation when compared to other studies²⁵, which can likely be attributed to the presence of NaF in solution. When comparing these results with the predicted dissolution rate of silica using models available in literature²⁵, NaF appears to increase degradation at 37° C. by a factor of 24 and degradation at 77° C. by a factor of 50.

Gate Leakage Current, SiO₂ vs SiC:

To test the performance of SiC as a dielectric insulator, we performed a gate leakage current study where we compared the chip to an identical chip that had SiO₂ instead of SiC as a dielectric layer. Silicon dioxide and other metal oxides have been for a long time the most commonly used gate dielectric in solid electronics, both for performance and ease of fabrication²⁶. However, more inert materials such as SiC may perform better in aqueous environments. The employed membranes have dielectric layers of comparable thickness (˜60 nm) and a buried conductive polySi layer used as electrode.

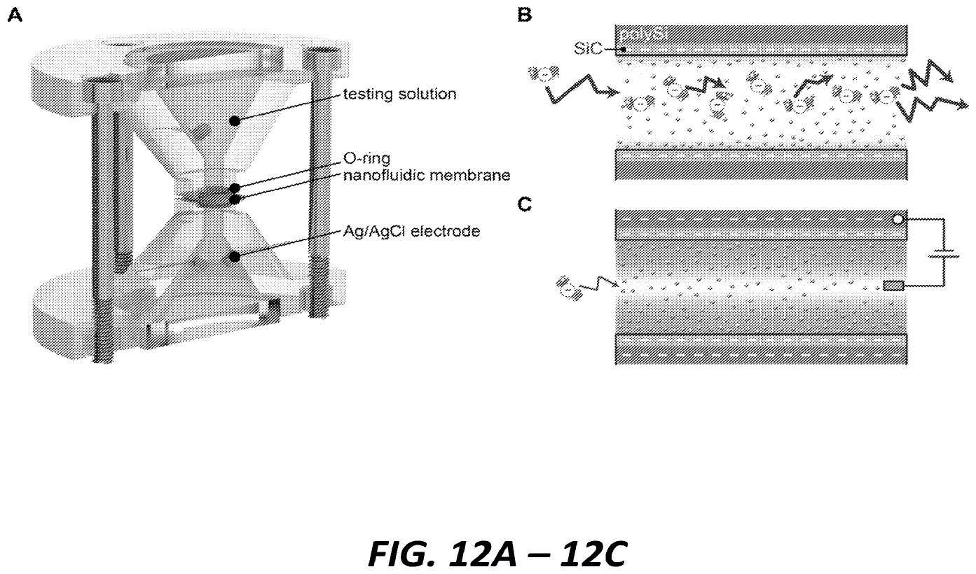

The leakage current is obviously affected by the external conditions, in particular the thickness of the insulating layer and the ionic strength of the solution. A thicker insulating layer results in a higher resistance and a lower current. Charged species in solution also affect the leakage current, a higher ionic strength leads to higher current due to ion infiltration in the insulating layer²⁵. In fact, the results show this linear dependence of the leakage current with the ionic strength of the solution (FIG. 11A, 11B) for both SiO₂ and SiC. The reason for the measured leakage currents at low voltages and its proportionality with the ionic strength stands in the non-ideality of the insulating materials.

Although SiO₂ and SiC have high intrinsic breakdown voltages, ˜15 MV/cm²⁸ and ˜2 MV/cm²⁹ respectively, leakage currents were measured in the order of tens and hundreds of μA in these nanofluidic membranes with just 0.5 MV/cm. In fact, the presence of defects and irregularities both in the oxide layer (dust particles) or at the Si—SiO₂ interface can increase the current flow at low electric field³⁰. Although this phenomena has been investigated for more than 50 years, several aspects of the time-dependent dielectric breakdown are not yet fully understood²⁸. Nonetheless, some of the steps involved are usually agreed upon: with the application of an external electric field, electrons are injected and trapped into the oxide triggering material degeneration. The random point defects generated throughout the oxide film can lead to a cluster of defects within tunneling distance that connect both sides of the film facilitating electron flow. This model is call percolation model and the resulting percolating path also known as conductive filament³¹ leads to increased currents through the insulating films³².

In aqueous solutions, the creation of defects can be accelerated by the migration of hydrogen in the form of protons in the insulating material³³. In this situation the dissolution of a percolating path in the dielectric film can create nanometric pores due to the changed stoichiometry of the insulating layer³⁴. Therefore, the higher ionic strength of the solution results in a higher probability of defect creation and thus higher recorded currents.

It is interesting to notice that the current for negative applied voltages is significantly higher than the respective positive voltages. There are at least two possible reasons for this asymmetry. First is the n-type doping of the polySi which inevitably suffers from polydepletion, which is known to be a downside for polysilicon gates in CMOS transistors because it can reduce the drain current and affect overall the performance of a solid-state transistor³⁵. The second is related to the formation of conductive filaments inside the insulating material. With the application of a negative voltage, the electric field generated pushes the protons from the solution to solid interface. When they reach the insulator/polySi interface they can exchange the electron. This mechanism stimulates the creation of the conductive filament in the insulating material resulting in high currents. On the other hand, the application of a positive voltage, pushes the protons toward the solution, resulting in the breaking of eventual conductive filaments already present. This results in a significantly lower leakage current.

Both materials seem to be affected by these phenomena, therefore it was decided to adopt nanofluidic membranes coated with SiC due to greater reliability in aqueous environments.

Mechanism of Electrostatically Gated Diffusion in Nanochannels:

Molecular diffusion in nanoconfinement exhibit peculiar phenomena which are typical of the nanoscale. Charged particles electrostatically interact with the charged surfaces of the nanochannels walls creating an ionic distribution know as electric double layer (EDL). The EDL can extend for several nanometers depending on parameters such as the ionic strength of the solution and the surface charge. This spatial extent of electrostatic interaction with the wall has a characteristic dimension called Debye length. In the EDL region depending on the sign of the surface charge, the concentration of charged molecules can either increase of decrease with respect to the bulk, to balance the surface charge and bring the nanochannel to electrostatic equilibrium. In this specific case, SiO₂ exposes negative silanol (SiO⁻) groups when in aqueous solution (pH 7.4) resulting in a net negative charge at the solid/liquid interface³⁶. Albeit in smaller density, SiC also exposes negative silanol groups resulting in an interface behavior similar to SiO₂^{37,38}.

Every charged molecule that electrostatically interacts with the surface charge will experience an increase or decrease in concentration dictated by the Poisson-Boltzmann distribution of potential at the interface³⁹. Negative molecules in this case will be repelled by the negative surface charge, therefore their overall concentration in the nanochannel will also be reduced. Thus, resulting in a net reduced diffusive flow with respect to a completely neutral channel. For this reason, the control over the surface charge allows the indirect control of the apparent diffusivity of charged molecules in nanoconfined space. The surface charge could in theory be increased to completely prevent a co-ion from entering the channel, creating a gate.

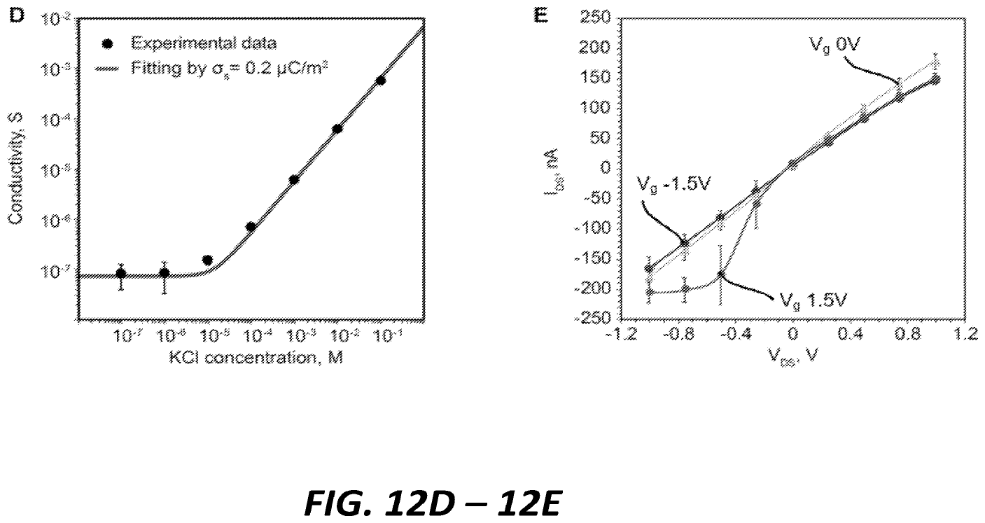

Here, the surface charge at the SiC/electrolyte interface is modulated by applying a potential between the poly-Si electrode and the electrolyte solution in a custom-made fixture (FIG. 12A). When no voltage is applied, the reduced extent of the EDL, allows for free diffusion of a negative molecule (FIG. 12B). In contrast, the application of a negative potential results in an increase in EDL extension and repulsion of negatively charged molecules from the channel volume (FIG. 12C). The diffusive flow rate of negatively charged molecules across the membrane was compared when different voltages are applied to the gate poly-Si electrode, with the expectation of a direct proportionality between the intensity of the applied voltage and the reduction in molecular diffusion.

Electrochemical Characterization of the Nanofluidic Membrane:

To test the capability of the membrane to modulate the release of charged molecules leveraging electrostatic gating, the membrane conductivity at different concentrations and the I-V response was investigated. An hourglass shaped fixture made of PMMA was designed that features two reservoirs that can be easily washed and replenished (FIG. 12A). The membrane under investigation is clamped between the reservoirs using gaskets to both avoid leakage and limit the chip surface exposure to the liquid to only the nanochannels area.

Ionic conductance through the membrane can be ideally separated in bulk conductance and surface dominated conductance⁴⁰. For high concentrations, the nanochannel height over Debye length ratio is h/λ>>1, therefore the measured conductance is consistent with bulk electrolyte conductance hence proportional to the ionic strength. Whereas for h/λ˜1 or h/λ<1 where the Debye length is comparable with the channel characteristic dimension, the conductivity becomes independent of the ionic strength and the channel height. In fact, the ions in the channels are mostly counter-ions that balance the surface charge to achieve electroneutrality, resulting in a conductance that only depends on the surface charge.

For these membranes the transition between bulk and surface dominated conductance happens for a 10 μM KCl solution (FIG. 12D), where the Debye length is expected to be ˜200 nm. The experimental results are in good agreement with the know conductance equation⁴⁰:

$$\frac{I}{V} = 2 F μ \sqrt{{( \frac{Σ}{2} )}^{2} + {c}_{0}^{2}} \frac{w h}{l}$$

where F is Faraday's constant, μ is the ionic mobility, Σ is the molar concentration of ions in the nanochannel volume, c₀ is the molarity of the solution, and w, h, and l are respectively the width, height, and length of the nanochannel. Σ was determined fitting the experimental data, resulting in a surface charge σₛ=0.2 μC/m² obtained using the relation zFΣ=−2σₛ/h and consistent with what was previously observed⁴¹.

Additionally, I-V curve measurements were performed to assess the gating capabilities of the membrane. A transmembrane voltage (V_{DS}) and a gate voltage (V_{GS}) were applied with the intent to investigate how the transmembrane current IDS is affected by the application of a gate voltage. FIG. 12E shows the representative I-V curves obtained with a 10 μM KCl solution and different V_{GS} applied. A clear dependence of the transmembrane current with the gate voltage can be seen. Specifically, an increase was seen in conductance with the application of a positive gate potential, especially for negative transmembrane voltages. Although unusual for SiO₂ nanochannels, where the conductance decrease with the application of a positive gate voltage, it has been previously reported and attributed to a slip flow at the wall⁴². This phenomenon happens for SiO₂ channels when the external V_{DS} is intense enough to overcome the attraction of the Stern layer K⁺ ions to the silica surface and move the Stern layer tangential to the surface. This can explain the behavior that was observed because these nanochannels have a SiC surface that exhibits a significantly reduced surface charge when compared to SiO₂.

In Vitro Release Rate Modulation of Alexa Fluor 647:

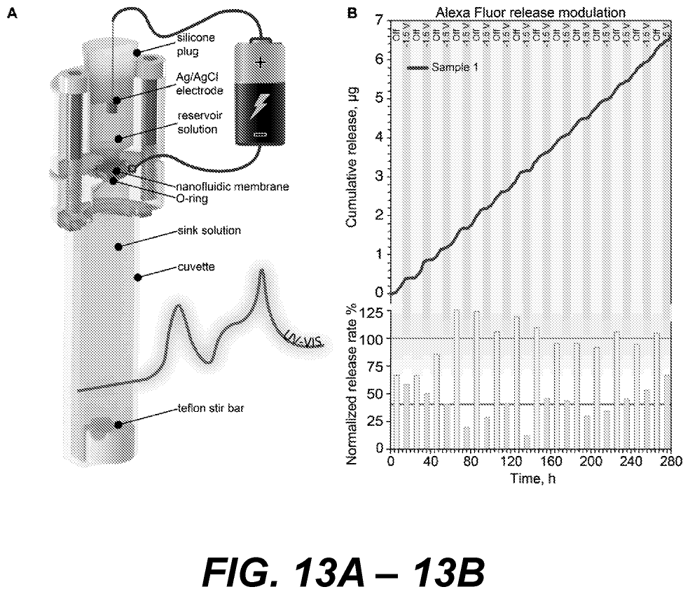

As a proof of concept of diffusion modulation of a charged particle, the influence of a negative gate voltage on the release of Alexa Fluor 647 (AF647), which is a commonly used fluorescent dye, was investigated. The release was performed in a custom-made release fixture that features a reservoir that contained a high concentration of AF647 and a sink reservoir with 0.01×PBS (FIG. 13A). The nanofluidic membrane is clamped between the two reservoirs and connected to an external voltage generator (represented as battery). Two release phases were alternated: a passive phase where no voltage was applied (0 V) and an active phase where a negative (˜1.5 V) was applied.

FIG. 13B shows the cumulative release rate of AF647, which exhibit a net charge of −3q when in PBS solution at pH 7.4. The blue and red columns represent the passive (12 h) and active (8 h) phases respectively. During the passive phases the molecules are released following a concentration driven diffusion, achieving a constant release rate. Upon application of the active phase, the increase surface charge effectively repels co-ions from the nanochannels, AF647 included, reducing its concentration and thus overall diffusion rate. In fact, the release rate during the active phases is consistently reduced with respect to the previous phase.

For ease of comparison, the release rate of each phase calculated form the slope of the cumulative release is plotted in the bar graph in FIG. 13B (bottom). To quantify the effect of the gating the release rate of each phase (horizontal lines) were averaged and compared the active to the passive release. A 60% reduction of release rate was observed during the active phases. Moreover, when averaging the release rate of each group, a statistically significant difference was detected, showing effective and repeatable release rate modulation leveraging electrostatic gating.

In Vitro Release Modulation of PolyStyrene Sulfonate:

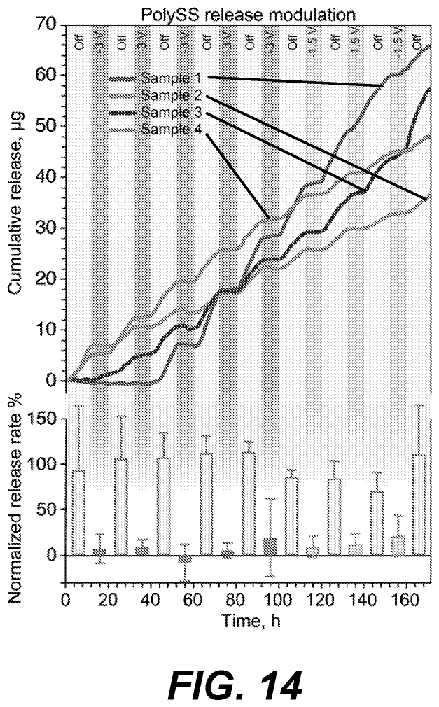

To better evaluate the modulation capabilities of the membrane and moreover how these change with different gate voltages, an in vitro release study was performed with Poly(sodium 4-styrenesulfonate), which is a highly charged polymer. FIG. 14 shows the cumulative release of PolySS when alternating passive (blue; labeled “Off”) and active phases (brown or red; labeled “−3 V” and “−1.5 V”). During the first 5 active phases (brown) a voltage of −3 V was applied to the gate electrode, while during the last 3, the voltage was reduced to −1.5 V.

As for AF647, the passive phases resulted in a sustained release for all the samples, whereas the application of a gate voltage consistently decreased the release rate. In particular, for −3 V the release rate was almost completely stopped in several occasion. Upon application of −1.5 V instead, the release rate was considerably reduced, but not stopped. The slope of each cumulative release was calculated and then normalized to the passive release rate (FIG. 14, bottom). A significant difference can be observed between the averaged release rates of the active and passive phases.

Importantly, the reduction and restoring of the release upon change of the applied voltage is consistently repeated, demonstrating that electrostatic gating performances do not degrade overtime.

In Vitro Release Modulation of DNA Salt:

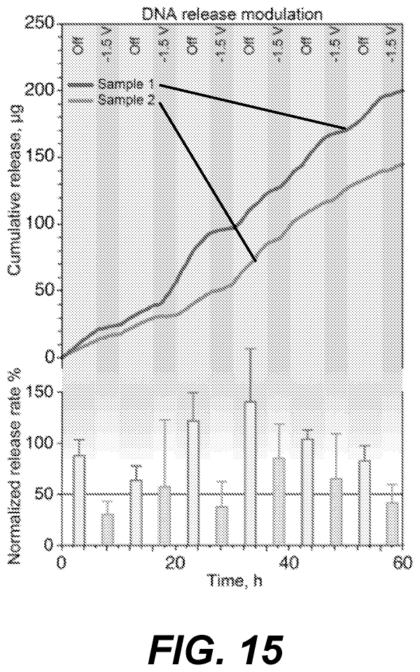

To demonstrate the controlled delivery of gene therapeutics, a release study was performed with DNA salt as a surrogate for plasmid DNA (pDNA) or small interfering RNA (siRNA). pDNA and siRNA are the two main vectors used in gene therapy for the treatment of incurable diseases such as cancer or various genetic disorder. FIG. 15 shows the cumulative release of DNA when alternating passive (blue; Off) and active phases (red; −1.5 V). As for the AF647 and polySS, the application of a negative voltage (−1.5 V) led to a substantial decrease of release rate with respect to the passive phase. Release rate analysis and normalization to the passive phases (bottom of FIG. 15) resulted in an average reduction of release rate of 50% of the passive release (bottom horizontal line). The passive phase yielded an average release rate of 89 μg per day. A target therapeutic dose for siRNA cannot be clearly identified, in part due to the fact that gene silencing therapies are still under development. However, this result provides confidence of the ability of the system to function in conjunction with biologics, and control molecular transport at rates that are within the same order of magnitude of experimental therapies.

Performance of Release Modulation Trough Electrostatic Gating:

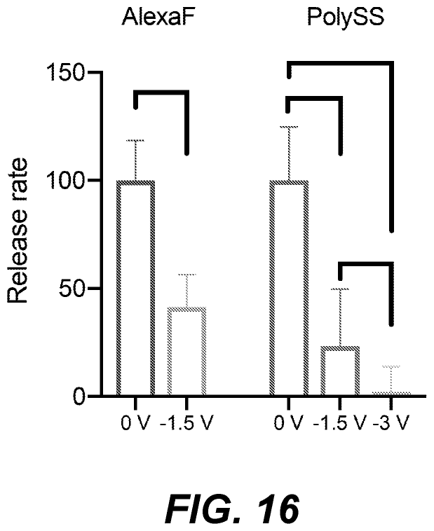

To better demonstrate the performance of electrostatic gating, FIG. 16 shows the normalized release rates of the molecules employed in this study, when grouped by applied voltage. For AF647 the application of −1.5 V resulted in a statistically significant reduction of release rate of ˜60%. Similarly, for PolySS both the applied voltages in the active phases yielded a statistically significant reduction in release rate when compared to the passive rate. Specifically, a release rate reduction of 77% was observed for −1.5 V and a remarkable 98% for the gate voltage of −3 V. Additionally, for PolySS a statistically different release rate reduction was observed between the two active phases. This result demonstrates the direct correlation between the intensity of the applied gate voltage and the reduction of release rate. In fact, higher gate electrode voltages lead to greater charge density at the liquid solid interface which result in a more extended EDL. When the EDL completely covers the nanochannel volume, co-ions are strongly repelled from the nanoconfined space, resulting in an almost complete stop of molecular diffusion.

Electrostatic Gating Energy Efficiency:

Power consumption ranging from 1.5 μW to 45 μW was measured depending on the applied voltage. Accordingly, commercially available and implant compatible 200 mA h batteries could support implant autonomy from 6 months to a few years, depending on the schedule of applied voltages. This represents a reduction in power consumption of nearly a magnitude over previous work^{43,44}, likely made possible by the adoption of electrostatic gating as opposed to electrophoresis or ionic concentration polarization, which were associated with substantially higher currents. In electrostatic gating the energy consumption is determined by leakage currents through the dielectric film. Materials such as high-k dielectrics can achieve very low leakage currents. However, they lack biocompatibility and bioinertness. SiC was chosen here because it showed exceptional bioinertness and achieved power consumptions comparable to previously developed gating devices.

Additionally, is important to notice the direct relation between the gating performance and the charge of the molecule employed. It is straightforward that the higher the charge, the more the molecule will be repelled by a charged surface of the same polarity. This difference can be noticed in the experimental results, in fact, with the application of −1.5 V, PolySS yielded a more pronounced reduction in release rate compared to AF647 mostly due to their difference in net carried charge.

Electrostatic gating is an efficient and reliable method to control the release rate of molecule across nanofluidic membranes. The ability to reversibly control the permeability of a nanofluidic membrane with the simple application of an external voltage renders this technology an ideal actuator for drug delivery. The proportional response between the applied voltage and the intensity of the release rate reduction offers facile implementation for the design of a platform that could offer fine and reliable control over drug release. Moreover, this technology could also offer a complete stop of molecule release if the employed voltages are greater than the ones demonstrated here.

Therefore, this platform for controlled release represents an efficient and highly controllable actuator for therapeutic administration systems. In this scenario, the membrane is connected to a control circuitry that autonomously, under remote control or with a pre-established schedule, can deliver therapeutics in dosages and timings that are completely built around the patient. Various strategies for power sourcing can include an external battery, an internal battery, inductive rechargeable batteries, and/or energy harvesting from the human body. In fact, it could be integrated with a completely implantable platform that provides a low-intensity voltage such as a battery, avoiding constant external energy supply. More than that, this technology can be leveraged as a drug delivery actuator in the next generation of closed-loop drug delivery systems which can offer true personalized medicine.

While the invention has been described with reference to particular embodiments and implementations, it will understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

### REFERENCES

1 Dugger, S. A., Platt, A. & Goldstein, D. B. Drug development in the era of precision medicine. Nat Rev Drug Discov 17, 183-196, doi:10.1038/nrd.2017.226 (2018).
2 Mujeeb, U. R. M., Nazari, M. H., Sencan, M. & Antwerp, W. V. A Novel Needle-Injectable Millimeter scale Wireless Electrochemical Glucose Sensing Platform for Artificial Pancreas Applications. Sci Rep 9, 17421, doi:10.1038/s41598-019-53680-7 (2019).
3 Taylor, M. J. et al. Closed-loop glycaemic control using an implantable artificial pancreas in diabetic domestic pig (Sus scrofa domesticus). Int J Pharmaceut 500, 371-378, doi:10.1016/j.ijpharm.2015.12.024 (2016).
4 Farina, M., Alexander, J. F., Thekkedath, U., Ferrari, M. & Grattoni, A. Cell encapsulation: Overcoming barriers in cell transplantation in diabetes and beyond. Adv Drug Deliv Rev 139, 92-115, doi:10.1016/j.addr.2018.04.018 (2019).
5 Yasin, M. N., Svirskis, D., Seyfoddin, A. & Rupenthal, I. D. Implants for drug delivery to the posterior segment of the eye: a focus on stimuli-responsive and tunable release systems. J Control Release 196, 208-221, doi:10.1016/j.jconre1.2014.09.030 (2014).
6 Compton, W. M. & Volkow, N. D. Improving Outcomes for Persons With Opioid Use Disorders Buprenorphine Implants to Improve Adherence and Access to Care. Jama-J Am Med Assoc 316, 277-279, doi:10.1001/jama.2016.8897 (2016).
7 Osterberg, L. & Blaschke, T. Drug therapy—Adherence to medication. New Engl J Med 353, 487-497, doi:DOI 10.1056/NEJMra050100 (2005).
8 Burkhart, P. V. & Sabate, E. Adherence to long-term therapies: Evidence for action. J Nurs Scholarship 35, 207-207 (2003).
9 Pons-Faudoa, F. P., Ballerini, A., Sakamoto, J. & Grattoni, A. Advanced implantable drug delivery technologies: transforming the clinical landscape of therapeutics for chronic diseases. Biomed Microdevices 21, doi:ARTN 47 10.1007/s10544-019-0389-6 (2019).
10 Heikenfeld, J. et al. Wearable sensors: modalities, challenges, and prospects. Lab Chip 18, 217-248, doi:10.1039/c71c00914c (2018).
11 Lin, H. et al. A rapid and low-cost fabrication and integration scheme to render 3D microfluidic architectures for wearable biofluid sampling, manipulation, and sensing. Lab Chip 19, 2844-2853, doi:10.1039/c91c00418a (2019).
12 Scholten, K. & Meng, E. A review of implantable biosensors for closed-loop glucose control and other drug delivery applications. Int J Pharm 544, 319-334, doi:10.1016/j.ijpharm.2018.02.022 (2018).
13 Merchant, F. M., Dec, G. W. & Singh, J. P. Implantable sensors for heart failure. Circ Arrhythm Electrophysiol 3, 657-667, doi:10.1161/CIRCEP.110.959502 (2010).
14 Kim, J., Campbell, A. S., de Avila, B. E. & Wang, J. Wearable biosensors for healthcare monitoring. Nat Biotechnol 37, 389-406, doi:10.1038/s41587-019-0045-y (2019).
15 Hoare, T. et al. Magnetically triggered nanocomposite membranes: a versatile platform for triggered drug release. Nano Lett 11, 1395-1400, doi:10.1021/n1200494t (2011).
16 Timko, B. P. et al. Near-infrared-actuated devices for remotely controlled drug delivery. Proc Natl Acad Sci USA 111, 1349-1354, doi:10.1073/pnas.1322651111 (2014).
17 Kim, K. et al. Externally controlled drug release using a gold nanorod contained composite membrane (vol 8, pg 11949, 2016). Nanoscale 8, 18810-18810, doi:10.1039/c6nr90236g (2016).
18 Ferrara, K. W. Driving delivery vehicles with ultrasound. Adv Drug Deliver Rev 60, 1097-1102, doi:10.1016/j.addr.2008.03.002 (2008).
19 Lee, S. H. et al. Implantable multireservoir device with stimulus-responsive membrane for on-demand and pulsatile delivery of growth hormone. Proc Natl Acad Sci USA 116, 11664-11672, doi:10.1073/pnas.1906931116 (2019).
20 Geninatti, T., Small, E. & Grattoni, A. Robotic UV-Vis apparatus for long-term characterization of drug release from nanochannels. Measurement Science and Technology 25, 027003 (2014).
21 Scorrano, G. et al. Gas Flow at the Ultra-nanoscale: Universal Predictive Model and Validation in Nanochannels of Angstrom-Level Resolution. ACS Appl. Mater. Interfaces 10, 32233-32238, doi:10.1021/acsami.8b11455 (2018).
22 Kanduti, D., Sterbenk, P. & Artnik, B. FLUORIDE: A REVIEW OF USE AND EFFECTS ON HEALTH. Mater Sociomed 28, 133-137, doi:10.5455/msm.2016.28.133-137 (2016).
23 Lee, Y. K. et al. Dissolution of Monocrystalline Silicon Nanomembranes and Their Use as Encapsulation Layers and Electrical Interfaces in Water-Soluble Electronics. ACS Nano 11, 12562-12572, doi:10.1021/acsnano.7b06697 (2017).
24 Zhou, D., Mech, B. & Greenberg, R. Accelerated corrosion tests on Silicon wafers for implantable medical devices. Vol. 363 (2000).
25 Crundwell, F. K. On the Mechanism of the Dissolution of Quartz and Silica in Aqueous Solutions. ACS Omega 2, 1116-1127, doi:10.1021/acsomega.7b00019 (2017).
26 Robertson, J. High dielectric constant gate oxides for metal oxide Si transistors. Rep. Prog. Phys. 69, 327-396, doi:10.1088/0034-4885/69/2/R02 (2005).
27 Wang, H., Gao, J., Chen, Z. & Wu, W. in 2014 12th IEEE International Conference on Solid-State and Integrated Circuit Technology (ICSICT). 1-3.
28 Padovani, A., Gao, D. Z., Shluger, A. L. & Larcher, L. A microscopic mechanism of dielectric breakdown in SiO₂ films: An insight from multi-scale modeling. Journal of Applied Physics 121, 155101, doi:10.1063/1.4979915 (2017).
29 Oliveros, A., Guiseppi-Elie, A. & Saddow, S. E. Silicon carbide: a versatile material for biosensor applications. Biomed Microdevices 15, 353-368, doi:10.1007/s10544-013-9742-3 (2013).
30 Verweij, J. F. & Klootwijk, J. H. Dielectric breakdown I: A review of oxide breakdown. Microelectronics Journal 27, 611-622, doi:10.1016/0026-2692(95)00104-2 (1996).
31 Yao, J., Zhong, L., Natelson, D. & Tour, J. M. In situ imaging of the conducting filament in a silicon oxide resistive switch. Scientific Reports 2, 1-5, doi:10.1038/srep00242 (2012).
32 Tung, C. H. et al. Percolation path and dielectric-breakdown-induced-epitaxy evolution during ultrathin gate dielectric breakdown transient. Appl. Phys. Lett. 83, 2223-2225, doi:10.1063/1.1611649 (2003).
33 Godet, J. & Pasquarello, A. Proton Diffusion Mechanism in Amorphous SiO2. Phys. Rev. Lett. 97, 155901, doi:10.1103/PhysRevLett.97.155901 (2006).
34 Yun, J. et al. Dielectric Breakdown and Post-Breakdown Dissolution of Si/SiO₂ Cathodes in Acidic Aqueous Electrochemical Environment. Scientific Reports 8, doi:10.1038/s41598-018-20247-x (2018).
35 Arora, N. D., Rios, E. & Cheng-Liang, H. Modeling the polysilicon depletion effect and its impact on submicrometer CMOS circuit performance. IEEE Transactions on Electron Devices 42, 935-943, doi:10.1109/16.381991 (1995).
36 Behrens, S. H. & Grier, D. G. The charge of glass and silica surfaces. The Journal of Chemical Physics 115, 6716-6721, doi:10.1063/1.1404988 (2001).
37 Abro, D. M. K., Dablé, P., Girault, H., Amstutz, V. & Cortez-Salazar, F. Characterization of Surface State of Inert Particles: Case of Si and SiC. Journal of Minerals and Materials Characterization and Engineering 4, 720-726, doi:10.4236/jmmce.2016.41007 (2016).
38 Cogan, S. F., Edell, D. J., Guzelian, A. A., Ping Liu, Y. & Edell, R. Plasma-enhanced chemical vapor deposited silicon carbide as an implantable dielectric coating. J Biomed Mater Res A 67, 856-867, doi:10.1002/jbm.a.10152 (2003).
39 Schoch, R. B., Han, J. & Renaud, P. Transport phenomena in nanofluidics. Reviews of Modern Physics 80, 839-883, doi:10.1103/RevModPhys.80.839 (2008).
40 Yossifon, G., Mushenheim, P., Chang, Y.-C. & Chang, H.-C. Nonlinear current-voltage characteristics of nanochannels. Phys. Rev. E 79, 046305, doi:10.1103/PhysRevE.79.046305 (2009).
41 Grosjean, A., Rezrazi, M. & Tachez, M. Study of the surface charge of silicon carbide (SIC) particles for electroless composite deposits: nickel-SiC. Surface and Coatings Technology 96, 300-304, doi:10.1016/50257-8972(97)00180-1 (1997).
42 Vermesh, U. et al. Fast nonlinear ion transport via field-induced hydrodynamic slip in sub-20-nm hydrophilic nanofluidic transistors. Nano letters 9, 1315-1319 (2009).
43 Bruno, G. et al. Leveraging electrokinetics for the active control of dendritic fullerene-1 release across a nanochannel membrane. Nanoscale 7, 5240-8 (2015).
44 Trani, N. et al. Remotely controlled nanofluidic implantable platform for tunable drug delivery. Lab Chip 13, 2192-2204 (2019).
45 Trani, N. et al. Electrostatically gated nanofluidic membrane for ultra-low power controlled drug delivery. Lab Chip 20, 1562-1576 (2020).

What is claimed is:

1. A device for controlling molecular transport, the device comprising;
a membrane comprising a plurality of nanochannels extending therethrough, an inner electrically conductive layer, and an outer dielectric layer, the dielectric layer creating an insulative barrier between the electrically conductive layer and the contents of the nanochannels,
at least one electrical contact region positioned on a surface of the membrane and exposing the electrically conductive layer of the membrane for electrical coupling to external electronics, and
wherein, when the membrane is at a first voltage, molecules flow through the nanochannels at a first release rate, and
wherein, when the membrane is at a second voltage, charge accumulation within the nanochannels modulates the flow of molecules through the nanochannels to a second release rate that is different than the first release rate.

2. The device of claim 1, further comprising a handle layer positioned beneath the membrane, the handle layer comprising at least one macrochannel extending therethrough and fluidically coupled to the plurality of nanochannels of the membrane.

3. The device of either claim 1 or claim 2, wherein edges of the dielectric layer define a gap in the dielectric layer that exposes the electrically conductive layer at the electrical contact region.

4. The device of any one of claims 1-3, wherein each nanochannel comprises an outlet on an upper surface of the membrane and an inlet connected to a macrochannel.

5. The device of any one of claims 1-4, wherein a height of a nanochannel, defined between a nanochannel inlet and a nanochannel outlet, is from 10,000 nanometers to 15,000 nanometers.

6. The device of any one of claims 1-5, wherein a length of a nanochannel, measured along a surface of the membrane, is from 400 nanometers to 5,000 nanometers.

7. The device of any one of claims 1-6, wherein a width of a nanochannel, measured along a surface of the membrane, is from 50 nanometers to 400 nanometers.

8. The device of any one of claims 1-7, wherein the dielectric layer resists degradation under physiological conditions.

9. The device of any one of claims 1-8, wherein the dielectric layer comprises a metal oxide.

10. The device of any one of claims 1-9, wherein the dielectric layer comprises silicon carbide.

11. The device of any one of claims 1-10, wherein the electrode layer comprises poly-silicon.

12. The device of any one of claims 1-11, wherein the macrochannels are hexagonal in shape.

13. The device of claim 12, wherein the macrochannels are arranged in a honey-comb pattern.

14. The device of any one of claims 1-13, wherein, when submerged in a physiological solution, the current leakage of the device is less than 300 microamps when the voltage applied to the electrical contact region is from −1V to −3V.

15. The device of any one of claims 1-14, wherein, when submerged in a physiological solution, the device has ultra-low power consumption.

16. The device of any one of claims 1-15, wherein the value of a voltage applied to the membrane at the electrical contact region determines the release rate.

17. A method of fabricating a device for controlling molecular transport, the method comprising:
etching a plurality of nanochannels through a membrane layer;
etching a plurality of macrochannels through a handle layer positioned below the membrane layer;
creating fluidic couplings between the macrochannels and the nanochannels;
applying a dielectric layer to the membrane layer and insulating the interior walls of the nanochannels with the dielectric layer; and
forming an electrical contact region that exposes an electrically conductive surface of the membrane layer.

18. The method of claim 17, wherein etching a plurality of nanochannels through a membrane layer further comprises etching through a membrane layer from an upper surface to a buried oxide layer that is positioned between the membrane layer and the handle layer, and wherein etching a plurality of macrochannels through a handle layer further comprises etching through the handle layer from a lower surface to the buried oxide layer, and wherein creating fluidic couplings between the macrochannels and the nanochannels further comprises removing the buried oxide layer.

19. The method of either claim 17 or claim 18, wherein the membrane layer comprises a silicon electrically conductive layer and the dielectric layer comprises silicon oxide.

20. The method of any one of claims 17-19, further comprising applying an electrically conductive layer to the membrane layer including the interior walls of the nanochannels prior to applying the dielectric layer to the membrane layer.

21. The method of claim 20, wherein the electrically conductive layer comprises doped polysilicon.

22. The method of either claim 20 or claim 21, wherein the electrically conductive layer is applied using low pressure chemical vapor deposition.

23. The method of any one of claims 20-22, wherein the electrically conductive layer is applied using ALD.

24. The method of any one of claims 20-23, wherein the dielectric layer comprises silicon carbide.

25. The method of any one of claims 20-24, wherein the dielectric layer is applied by plasma enhanced chemical vapor deposition.

26. The method of any one of claims 17-25, further comprising patterning a nanochannel template onto a mask layer prior to etching a plurality of nanochannels through the membrane layer.

27. The method of any one of claims 17-26, wherein the nanochannels are etched using deep reactive ion etching.

28. The method of any one of claims 17-27, wherein the macrochannels are etched using deep reactive ion etching.

29. The method of any one of claims 17-28, wherein the macrochannels are etched using wet etching.

30. The method of any one of claims 17-29, wherein the electrical contact region is formed by partially removing the dielectric layer.

31. The method of claim 30, wherein forming the electrical contact region further comprises partially removing the dielectric layer by reactive ion etching.

32. The method of any one of claims 17-31, wherein the electrical contact region is formed by masking during the deposition of the dielectric layer to the membrane layer.

33. A method of controlling the delivery of a therapeutic substance through a membrane, the method comprising:
applying a voltage to a membrane, the membrane comprising a plurality of nanochannels extending therethrough, an inner electrically conductive layer, and an outer dielectric layer, the dielectric layer creating an insulative barrier between the electrically conductive layer and the contents of the nanochannels,
inducing charge accumulation within the nanochannels extending through the membrane,
modulating the rate by which a therapeutic substance is released through the nanochannels.

34. The method of claim 33, wherein modulating the release rate further comprises releasing the therapeutic substance on an automated schedule.

35. The method of either claim 33 or claim 34, wherein modulating the release rate further comprises releasing the therapeutic substance upon receipt of user input.

36. The method of any one of claims 33-35, wherein, when submerged in a physiological solution, the device has ultra-low power consumption.

37. The method of any one of claims 33-36, wherein the therapeutic substance is housed in at least one reservoir adjacent to the plurality of nanochannels, and application of a voltage to the membrane results in flow of the therapeutic substance from the reservoir through the nanochannels.

38. The method of claim 37, wherein the at least one reservoir is a macrochannel that is fluidically coupled to the nanochannels.

39. The method of any one of claims 33-38, wherein applying a voltage to a membrane comprises applying a voltage to an electrical contact region of the membrane.

40. The method of any one of claims 33-39, wherein the release rate is dependent upon the value of the voltage of the membrane.

41. The method of claim 40, wherein a voltage of −1.5V results in a release rate reduction of greater than 50%.

42. The method of either claim 40 or claim 41, wherein a voltage of −3V results in a release rate reduction of greater than 90%
